(19) United States
(12) Patent Application Publication
(10) Pub. No.: US 2008/0281350 A1
(43) Pub. Date: Nov. 13, 2008

(54) Aneurysm Occlusion Devices

(75) Inventors: Ivan Sepetka, Los Altos, CA (US); Greg Mirigian, Dublin, CA (US); Arindam Datta, Hillsborough, NJ (US); Steven Hochberg, New York, NY (US); Ricardo Aboytes, Palo Alto, CA (US); Trang Ngo, Milpitas, CA (US)

Correspondence Address: KING & SPALDING, 1185 AVENUE OF THE AMERICAS, NEW YORK, NY, 10036-4003, US

(73) Assignee: BIOMERIX CORPORATION, New York, NY (US)

(21) Appl. No.: 11/956,323

(22) Filed: Dec. 13, 2007

Related U.S. Application Data
(60) Provisional application No. 60/869,855, filed on Dec. 13, 2006.

(51) Int. Cl. A61M 29/02

(52) U.S. Cl. 606/200

(57) ABSTRACT

An implantable occlusion device for bridging the neck of an aneurysm comprises a biocompatible matrix. The device is movable between a compressed position prior to implantation and a generally concave or cup-shaped position following implantation. The device may comprise a frame having a plurality of elements. The frame elements have a first pre-deployment position generally parallel to a major axis of the delivery lumen, and a second post-deployment position spread radially from the major axis of the delivery lumen. The biocompatible matrix and/or the frame elements may also form or be manipulated to form a generally concave or cupped shape. The matrix can be porous or semiporous, such as a foam or a reticulated matrix. The occlusion device can be folded, twisted and/or stretched to adopt a narrow profile for loading into a coaxial delivery device and expand in place as it adopts its original shape on release. The device may be released or manipulated to a desired shape to occlude an aneurysm. Methods of using the implantable device are also provided.

This applications claims priority benefit under 35 U.S.C. § 119(e) of U.S. Provisional Patent Application 60/869,855, filed on Dec. 13, 2007, entitled “Invertable Expandable Device for Aneurysm Occlusion Devices,” which is incorporated herein by reference in its entirety.

The present invention is directed to the field of medical devices. Specifically, the present invention is directed to implantable occlusion devices for bridging the neck of an aneurysm or other vasculature in a patient, and methods for their use.

### BACKGROUND

Current methods of treating aneurysms are designed to fill the aneurysm lumen or sac by introducing medical devices, such as coils. These methods often require deployment of multiple coils to seal the aneurysm. Many of these devices suffer from the problems associated with device compaction, such as recanalization of the aneurysm.

There is an ongoing need for an improved method of treatment of an aneurysm that provides a seal of the neck of the aneurysm and thereby permits tissue re-growth leading to a permanent repair, and wherein the seal is not subject to re-canalization and consequent reemergence of the aneurysm.

### SUMMARY OF THE INVENTION

One aspect of the present invention provides an implantable occlusion device for bridging the neck of an aneurysm of a patient. The neck is the access of the aneurysm (or other vascular deformation) to the vasculature of the parent artery. The device includes a physiologically compatible matrix, with or without a support structure. The matrix may have (1) a preset shape memory and may be unsupported by any other structure; or (2) the support structure may be an integral part of the matrix, or may involve non-matrix elements, such as frame elements that cooperate with the matrix when in use.

Although an aneurysm can be occluded without an implant or without providing some kind of protection to the aneurysm neck, the inventive implantable device provides a number of advantages, as briefly discussed below.

The present invention advantageously provides a biological seal between the parent vessel and the aneurysm. The invention also helps prevent migration or leakage of embolic agents or packing materials from the aneurysm sac into the parent vessel. For example, loss of a liquid embolic agent during aneurysm filling in the absence of a seal can present a high-risk condition for the patient. Similarly, migration of a solid embolic agent such as a string or other elongate packing material from the aneurysm sac can potentially be dangerous. Aneurysm neck occlusion provides a large safety factor for both doctor and the patient resulting in significant reductions in operating room time for the procedure.

The present invention also provides aneurysm neck reconstruction by patching internally or bridging the entire surface area of the original parental artery or the cross section of the access opening to facilitate re-growth of tissue. In one embodiment, the invention provides a “straight line” neck reconstruction of the original parental wall, even for aneurysms with wide necks which are very difficult to treat with coils or other materials. The present invention provides a significant advantage over currently known implants in that the inventive implant allows for normal flow in the parental artery because it does not affect or change the size of the artery or obstruct or disrupt the flow of blood inside the artery.

Because of its geometry and surface area, the present invention provides a simple and permanent biological seal of the neck of an aneurysm, thereby facilitating and resulting in permanent occlusion with the tissue re-growth and preventing the future re-canalization or re-bleeding of the aneurysm. The invention thus helps prevent re-canalization after embolic agents have been used to occlude the aneurysm.

Although the inventive device is particularly suited for sealing aneurysms, the invention can also be used to occlude any kind of vasculature in a patient. For example, the invention can be used to occlude arterial or venous fistulas or to treat gastro-intestinal bleeding. Other applications of the present invention will be apparent to those of skill in the art.

In this specification, the terms “implant”, “device”, “implantable device”, “occlusion device”, and “implantable occlusion device”, unless qualified otherwise, are used to refer to the implant device according to one aspect of the present invention.

The terms “frame” and “support structure” may in context refer to a portion of the implant which supports the matrix. The frame may but need not have a plurality of frame elements, such as frame arms that may be joined at one or more loci. Each of the frame arms may have the same or different structure, as further discussed below. The frame may also include additional elements, such as a radiopaque marker to facilitate visualization of the frame with medical imaging equipment. The frame may provide structure and biased support for the implant. Alternatively, the frame may be structurally configured in a manner which does not provide biased support to the implant, but rather is used for deployment and/or imaging purposes. All such embodiments are encompassed by the present invention.

The term “cup-shaped”, with reference to the shape of the device when it is in one embodiment of a treatment configuration, or fitted within an aneurysm in a concave orientation having a degree of conformity to the interior of the aneurysm and refers to any concave and substantially non-spherical or truncated spherical or non-spherical shape that the device will adopt in a folded or expanded configuration. The cup shape comprises a hyper-extended or partially-extended umbrella-type geometry. The cup can have an elongated cup shape such as a wine flute, or have a more flat cup shape such as a champagne coupe or a disc with an upturned rim. Other examples of cup shapes include bowls and parabolas. Hemi-spherical or truncated ellipsoid or spherical variations are also possible. In addition, the cup shape may be uniform or non-uniform. Further, the internal volume defined by the cup shape may be partially filled. For example, the cup shape may comprise a flower-shape as shown in FIG. 9E, the internal volume of which is partially filled by a folded matrix. All such variations of the general cup shape are encompassed by the present invention without limitation.

Advantageously, the cup design allows the implant to accommodate itself in a large number of aneurysm morphologies. This accommodation reduces the sizing challenges that are typically associated with spherical designs which normally require custom sizes for every aneurysm. Accordingly, the present invention allows for much greater flexibility by the physician.

The term “tubular”, with reference to the shape of the device when it is in an expanded configuration refers to various hollow shapes having a solid circumference and a hollow center along a longitudinal axis. A cross-section of the tubular shape perpendicular to the longitudinal axis may comprise any two dimensional shape, which may be regular or irregular. For example, the cross-section of the tubular shape comprise a circle, an ellipse, a semi-circle, a polygon (e.g., a triangle, a quadrilateral, a rectangle, a square, a parallelogram, a pentagon, a hexagon, an octagon), a star, and the like. In addition, the tubular shape may be uniform or non-uniform, the device may have varying thickness and may have differently sized and/or shaped cross sections along a length of the longitudinal axis. All such variations of the general tubular shape are encompassed by the present invention without limitation.

In this regard, non-perfect placement of the implantable device within the aneurysm sac is acceptable as long as the device can seal or substantially seal the neck of the aneurysm. Accordingly, the physician does not need to precisely position the implantation device as it can expand to accommodate the size of the aneurysm. In addition, the support structure, frame elements or matrix can expand in different amounts to fit the aneurysm. For example, frame elements on one side of the implantable device can expand much more than the frame elements which are adjacent or diametrically opposite to provide a better fit to the aneurysm geometry.

Although this disclosure may make reference to aneurysms located on artery or parental artery walls, the invention is equally applicable to aneurysms located on venous walls. Therefore, the term “artery” or “parental artery” is to be understood as being interchangeable with “vein” or “parental vein”.

An embodiment of the present invention provides an implant for occluding the neck of an aneurysm. The implant comprises a first, convex orientation which facilitates delivery of the implant to the interior of the aneurysm, and a second, substantially concave orientation which the implant adopts when in use in the interior of the aneurysm. The term “convex” as used in describing the embodiments of the present invention includes shapes that are generally convex, which includes shape that may have local concavities or surface irregularities.

Preferably, the concave orientation has a degree of conformity to an interior surface of the aneurysm. Each orientation may or may not depend upon the structure of the implant frame.

One embodiment of the present invention provides an implantable occlusion device for bridging the neck of an aneurysm. The device comprises (a) a frame having a plurality of elements; and (b) a matrix coupled to the frame. The frame elements have a first pre-deployment position which is generally parallel to a major axis of the delivery lumen of a delivery device and have a first orientation, and a second post-deployment position in which the frame elements are extended or spread radially from the major axis of the delivery lumen and have a second orientation. The frame elements may be symmetrically or asymmetrically deployed in the aneurysm sac.

The first orientation may comprise a component direction substantially toward the proximal end of the lumen, and the second orientation may comprise a component direction substantially away from the proximal end of the lumen.

Another embodiment of the present invention comprises a frame and a matrix, wherein the frame has a first collapsed-umbrella position prior to implantation and a second hyperextended-umbrella position following implantation.

Another embodiment of the present invention provides an apparatus for occluding the neck of an aneurysm. The apparatus includes the following elements:

(a) an implantable occlusion device movable between a compressed position prior to implantation and a generally cup-shaped position following implantation; and

(b) a delivery device having a lumen, wherein the occlusion device is releasably mounted to a tip of the delivery device; and

(c) a coaxial detachment core wire located in the lumen of the delivery device.

The implantable occlusion device may comprise: a plurality of frame arms movable between a collapsed position prior to implantation and a generally cup-shape position following implantation and a matrix coupled to the frame arms; or a compressible matrix having a preset shape. The delivery device may be a catheter, microcatheter, or another device or instrument without limitation which can position the implant in the desired position in the vasculature. The apparatus may further comprise an embolic agent which facilitates occlusion of the aneurysm.

Another embodiment of the present invention provides an apparatus for occluding the neck of an aneurysm. The apparatus includes an implantable occlusion device comprising a compressible matrix having a tubular shape, the matrix having a plurality of slits. The matrix capable of folding into a cup-shape.

Another embodiment of the present invention provides a method for occluding an aneurysm. The method comprises the steps of:

(a) providing the inventive occlusion device, wherein the occlusion device is releasably attached to a delivery device having a lumen;

(b) positioning and deploying the occlusion device in the area of the aneurysm so that the device expands to or is manipulated to a cup-shape position and thereby bridges and seals the neck of the aneurysm;

(c) delivering an embolic agent through the lumen of the delivery device into the aneurysm to secure the occlusion device at the neck of the aneurysm; and

(d) detaching the delivery device from the occlusion device.

Another embodiment of the present invention provides a device for treating an aneurysm having an internal wall defining an internal volume. The device comprises an implant adaptable to (1) a collapsed configuration for delivery to the aneurysm, (2) an expanded configuration for delivery into the internal volume of the aneurysm, and (3) a treatment configuration having at least one region of increased diameter greater than the diameter of the collapsed configuration, the region of increased diameter having at least one proximal face and at least one distal face. The implant being further adaptable to assume a treatment position within the aneurysm, in which position the at least one proximal face is convex.

To avoid dissection, that is, a tear in the wall of an artery that causes blood leakage, the implant according to the present invention may be suitably sized such that, when fully expanded, the implant is approximately the same size in each dimension as the equivalent dimension of the aneurysm sac, and thus the implant fits snuggly into the aneurysm sac. Alternatively, the implantation device may be slightly larger or smaller in one or more dimensions than the aneurysm sac. Because the neck of the aneurysm is generally smaller than the diameter of the aneurysm sac, the expanded implant is secured and resists expulsion from the aneurysm. Furthermore, the implant is structurally designed and configured to be anchored in the neck of the aneurysm after delivery of an embolic agent into the sac of the aneurysm. The embolic agent ensures that the implant maintains a desired position at the neck of the aneurysm and allows for formation of a tight seal. As such, devices according to the present invention do not require any anchoring component extending into the parental artery.

In one embodiment of the invention, the implant has a width when expanded which is twice the size of the width of the aneurysm neck. In other embodiments, the implant has a width when expanded which is 110%, 125%, 140%, or 175% of the width of the aneurysm neck. Such embodiments provides an efficient capping of the aneurysm by locking the implant at the neck and preventing its ejection into the patient's vasculature.

Generally, the dimensions of the matrix material will be selected such that the matrix will cover or block the aneurysm opening or aneurysm neck after insertion of the occlusion device in the aneurysm. In a particular embodiment, the matrix of the device substantially seals the opening of the aneurysm. In another embodiment, the matrix of the device completely closes the opening or neck of the aneurysm. The implant may be circular, non-circular, elliptical, or other shapes, and can be tailored to the aneurysm neck geometry.

The implant will typically be delivered to the aneurysm in a compressed, collapsed or partly collapsed position via the patient's vasculature. For example, the implant can be folded and/or stretched on a guidewire or on an internal sheath (that may harbor a guidewire), in order to attain a cross section narrow enough to be preloaded into the delivery device, which may be a catheter or other instrument. After delivery in the aneurysm, the compressed, collapsed or partly collapsed implant is expanded to reach its full dimensions and if necessary, manipulated into a desired shape, and the delivery device is subsequently removed from the patient's body.

Although it is envisioned that one implant will be typically suffice to seal an aneurysm, in certain instances, a physician may wish to use a plurality of implants to seal the aneurysm. For example, the physician may wish to place two implants side by side in an interlocking arrangement to bridge the neck of the aneurysm. Such embodiments incorporating the use of a plurality of implants are within the scope of the present invention. In such embodiments, one or more implants can be delivered by the same delivery micro-catheter, or a plurality of delivery devices can be used in serial fashion to deliver the aneurysm-sealing devices before placing an embolic agent into the aneurysm.

The frame of the implant will generally be extremely flexible and can be constructed of any biocompatible material. For example, the frame may be a metallic frame. In one embodiment, the frame is prepared from nitinol. The matrix can be formed from any suitable substance known to those of skill in the art. Non-limiting examples of biodurable reticulated elastomeric matrices containing inter-connected and inter-communicating pores are described in the following U.S. patent applications: Ser. No. 10/749,742, filed on Dec. 30, 2003; Ser. No. 10/848,624, filed on May 17, 2005; and Ser. No. 10/998,357, filed on Nov. 26, 2004. All of these applications are assigned to Biomerix Corp. and are incorporated herein by reference in their entirety.

In one embodiment, the occlusion device of the present invention is a Neuro-Cup™ implantation system developed by Biomerix Corp.

Because the present invention can prepared in a number of sizes, it can be used to treat aneurysms in patients of any age, including infants and small children, adults, and the elderly. The physician will select the appropriately-sized occlusion device for each patient on a case-by-case basis.

The invention can also be used in any part of the body for treatment of aneurysms or for occlusion of vasculatures. For example, the invention can be used to treat medical conditions such as aneurysms in the brain, aorta, chest, and other parts of the body; facial tumors; arterial or venous diseases; blood vessel malformation; and hemangiomas.

The invention can be provided as a ready-to-use apparatus to the physician, or as components requiring final assembly by the physician.

The present invention furnishes a high level of control of the placement and detachment of the implant. For example, in the event that the initial placement of the implant is not suitable, in certain embodiments the implant can be withdrawn back into the delivery device by reversing the delivery process, i.e. by applying torque in the opposite direction to the direction of torque during the initial delivery attempt and collapsing the implant for repositioning. Non-suitable placement of the implant may occur, for instance, if the implant has been prematurely released, whether deliberately or accidentally, and partially expanded, but is either not accurately placed or has migrated into the parental artery from the initial delivery site. Withdrawal of the misplaced device allows for subsequent redeployment and even permits multiple attempts to accurately position and fit the aneurysm-sealing device to the desired location in difficult-to reach aneurysms.

Without being bound by any particular theory, it is believed that occlusion or sealing of the aneurysm by the present invention occurs first as the matrix acts as a mechanical barrier to reduce the flow of blood from the parent vessel into and out of the aneurysm sac. The matrix acts as a thrombotic patch and the stagnation of flow initiates a thrombotic response characterized by formation of a platelet-fibrin clot. This stage is followed by organization of the clot and finally, in the last stage of the healing response, resorption and resolution of the clot into fibrovascular tissue and thereby sealing the aneurysm.

The use of a reticulated matrix which is permeable to blood or other bodily fluids allows cells or other biological tissues to access the interior surfaces of the implant. The presence of inter-connected and inter-communicating reticulated open pores, voids, channels, and/or concavities in the matrix thus permits the formation of fluid passageways or fluid access into and out of the implant.

Accordingly, an embodiment of the present invention permits total reconstruction of the parental artery by delivering a patch of the physiologically-compatible matrix across the neck of the aneurysm, thereby providing a tissue scaffold to promote endothelial growth, or tissue growth and proliferation to form a biological seal. Sealing the opening or neck of the aneurysm results in permanent aneurysm occlusion and eliminates the risk of recanalization of the aneurysm sac. This approach also offers the advantage of one-time repair or “single-shot occlusion” by deployment of an appropriately-sized cup held in position by the expanded frame and an embolic agent contained therein to seal the aneurysm opening. As such, the expanded aneurysm-sealing device of the present invention has the potential to significantly reduce operating room time utilization, leading to significant economic advantages.

Other advantages and features of the invention will become apparent from the following discussion and figures.

This application is related to U.S. patent application Ser. No. 10/998,357, entitled “Aneurysm Treatment Devices and Methods”, filed on Nov. 26, 2004; and U.S. provisional patent application Ser. No. 60/785,901, filed on Mar. 24, 2006. The contents of both of these applications are incorporated herein by reference in their entirety.

### BRIEF DESCRIPTION OF THE FIGURES

The attached Figures depict embodiments of the invention and are intended for illustration purposes only. These Figures are not intended to be interpreted as limitations to the scope of the claimed invention.

FIG. 1A illustrates a first embodiment of the invention showing an implantable device in the collapsed state. FIGS. 1C and 1D respectively show the device in partially expanded and fully expanded positions in an aneurysm after passive deployment of the implant. FIG. 1B shows a cross-section of the delivery sheath illustrated in FIG. 1A.

FIG. 2A illustrates a second embodiment of the invention showing an implantable device in the collapsed state. FIGS. 2B and 2C show views of a section of the delivery sheath illustrated in FIG. 2A. FIGS. 2D and 2E show views of the implant of FIG. 2A in an expanded condition after active deployment of the implant.

FIGS. 3A-3E illustrate a method of delivering an embodiment of the inventive device having shape memory arms into an aneurysm, deploying the device using a passive opening of the umbrella and flipping into a cup shape, filling the aneurysm with an embolic agent, and detaching the device from the delivery device.

FIG. 4 illustrates an embodiment of the inventive device which is in the process of being filled with an embolic agent in the form of a string after active deployment.

FIGS. 5A-5C illustrate a double-hole coupling and detachment mechanism which may be used to facilitate delivery of the implantable device to the vasculature of a patient for treatment.

FIG. 6 illustrates several embodiments of frame elements according to an aspect of the present invention. The frame elements may be welded, hinged, or connected in some other manner to form the implant.

FIGS. 7A-7C illustrate an embodiment of the invention wherein the frame arms of the inventive device are joined at their proximal and peripheral ends to form a rosette cup after deployment.

FIG. 8A-8C illustrate various embodiments of a matrix having a tubular form capable of folding into a cup-shape which may be used in connection with the present invention.

FIGS. 9A-9E illustrate various steps of a method of delivering an embodiment of the inventive device having a tubular matrix into an aneurysm of a patient.

FIGS. 10A-10D illustrate two embodiments of a matrix folding mechanism which may be used to deliver an embodiment of the inventive device having a tubular matrix into an aneurysm of a patient.

FIGS. 11A-11B illustrate an embodiment of the inventive device having a conical matrix.

### DETAILED DESCRIPTION OF THE INVENTION

Features of the present invention will now be described with more particularity, including with reference to the Figures.

### Deployment and Sizing Considerations

The implantable device according to the present invention can be deployed, or moved from a collapsed position to an expanded position, in an aneurysm using active or passive means. Combinations of active and passive deployment are also possible and within the scope of the invention.

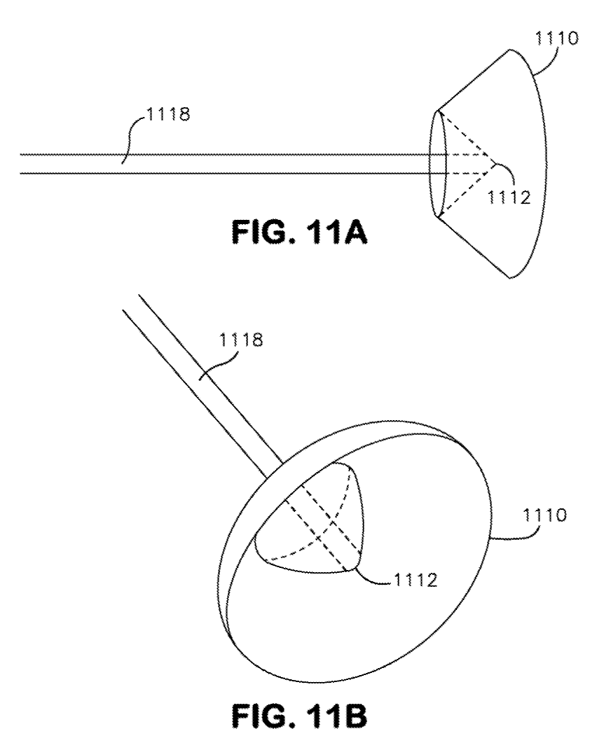

For passive deployment, the implant expands by assuming its preset shape memory after release from the delivery device. The implant may comprise a frame and/or a matrix having a preset shape memory. According to this embodiment, the device is delivered to the sac of the aneurysm in a collapsed or partially-collapsed position, and the implant is then released. For example, an implant having a frame comprising frame arms may be deployed from a compressed arrangement to the preset shape memory. Alternatively, the implant may comprise a matrix having a preset shape memory, which may be compressed for delivery and restored upon deployment. The implant subsequently opens from the collapsed or partially-collapsed condition to an expanded or extended position by the radial expansion force of the previously-compressed implant. Blood flowing in the sac of the aneurysm also urges the implant to extend upwards and outwards to adopt the implant's preset hyper-extended cup or reversed umbrella shape, or conical/dish-shape (as shown in FIGS. 11A and 11B).

Passive deployment may also involve the physician partially retracting or pulling back on the expanded or partially-expanded implant while it is in the sac. This movement allows certain embodiments of the implant to completely flip into the desired final cup shape inside the aneurysm sac and to be firmly secured to the walls of the aneurysm. The partial retraction or pulling action substantially flips the implant into the desired final shape, and forms at least a partial seal with the walls or neck of the aneurysm. Alternatively, the partial retraction or pulling action partially flips the implant and folds the implant into a desired final shape, which may provide a higher degree of conformity to the interior and/or neck of the aneurysm. This restricts access of the aneurysm to the vasculature by forming a biological or mechanical barrier.

After the frame has expanded and the device is in the desired position, embolic agents such as coils or glue are delivered through the lumen of the delivery device to anchor the implant in place. The implant is then detached from the delivery device.

For active deployment, the implantable device is actively manipulated in the sac into the desired cup shape by the physician using mechanical means. According to one embodiment of this feature, an implant having a frame is delivered to the sac of the aneurysm, and the frame is released. The delivery device is then mechanically pulled back and the frame is flipped back to form a cup shape within the sac. In an alternative embodiment of, the implantable device may comprise a matrix in a tubular form having a number of slits. As the delivery device is mechanically pulled back, a distal end of the tubular matrix is pulled towards a proximal end of the tubular matrix and the matrix is folded to a concave orientation having a degree of conformity to the interior of the aneurysm or to a cup-shape or flower shape within the sac. Both embodiments form at least a partial seal to the neck or restrict access of the aneurysm, and thereby, restricting access of the aneurysm to the vasculature by forming a biological or mechanical barrier.

The shape of the device may be unsupported by a separate structure (e.g., a frame) or may be at least partially supported by a frame. In one particular embodiment, the frame may comprise a plurality of frame arms, which can be flipped to form the cup shape within the sac when the device delivery device is mechanically pulled back.

Active deployment of the implantable device of the present invention does not depend on blood flow for formation of the final cup configuration, although the blood flow or the use of a shape memory material in the implantable device may assist in the inversion action or the folding of the matrix. To facilitate the inversion of the implant, e.g., in some embodiments via flipping of the frame arms to the deployed position, the attachment of the frame arms to the implant may incorporate a hinging mechanism. Different types of active deployment are possible and depend upon the specific structural configuration of the implant. It is envisioned that different implant designs may have different mechanisms for opening the implant into the cup shape.

Accordingly, the implantable device may be structurally designed and configured for passive deployment, active deployment, or a combination of both.

The particular shape and dimensions of the inventive occlusion device will depend on the size of the aneurysm to be treated, which can be readily determined by the physician using standard test procedures. For example, the physician may use a radiopaque dye to fill the aneurysm and aid in assessing its shape and dimensions. In certain instances, the aneurysm may be a vascular deformation.

Aneurysms are generally from about 2 mm to about 20 mm in the largest dimension or largest transverse dimension. Small aneurysms can be from about 2 mm to about 4 mm; medium sized aneurysms are generally from about 5 mm to about 9 mm in the largest dimension; and the largest aneurysms are generally from about 10 mm to about 20 mm in the largest dimension or their largest transverse dimension, although even larger aneurysms are not unknown.

The size of the implant chosen to repair a particular aneurysm will generally be approximately the same size as the aneurysm. In certain instances, the expanded implant may have a transverse diameter which is slightly smaller or slightly larger than the width of the aneurysm. The diameter of the implant will generally be substantially wider than the neck of the implant, and the shape of the device is generally chosen to most nearly match the shape of the aneurysm. Such sizing considerations will provide good fit between the aneurysm and the implant. However, in certain instances, a physician may deliberately choose a device which does not closely match the shape of the aneurysm.

In an embodiment of the invention, the implant in its concave orientation can be from about 2 mm to about 20 mm in diameter. In another embodiment, the device in its concave orientation can be from about 4 mm to about 15 mm in diameter. In still another embodiment, the device in its concave orientation can be from about 5 mm to about 10 mm in diameter, or from about 6 mm to about 8 mm in diameter. In other embodiments of the invention, the implant in its concave orientation will have an expanded cross-sectional or transverse diameter which is outside any of these ranges. It is estimated that 80% of aneurysms are between about 3 mm and about 10 mm in diameter.

In a particular embodiment, the present invention, when in its deliverable form, e.g. when compressed to fit into a delivery microcatheter, has an outer diameter of from about 2 French (i.e. 0.026 inch/0.67 mm) to about 5 French (i.e. 0.065 inch/1.7 mm). The deliverable implant, which is at least partially compressed or collapsed, even when loaded into a microcatheter, will generally maintain a high degree of flexibility so that the delivery device can be easily navigated through the vasculature to the intended area of treatment.

In certain embodiments, the implantable device may be compressed by applying a rotational force, e.g., twisting, the matrix portion of the implant. In one particular embodiment having a tubular matrix, the matrix may be compressed longitudinally. Specifically, the tubular matrix may be compressed by applying a rotational force to or twisting the tubular matrix along a longitudinal axis of the matrix.

In one embodiment, a first securing structure may be affixed at or near the distal end of the tubular matrix and a second securing structure may be affixed at or near the proximal end of the tubular matrix, wherein the first securing structure may be secured by a delivery device and the second securing structure can be manipulated by a physician during delivery of the implantable device. Alternatively, the second securing structure may be secured by the delivery device and the first securing structure can be manipulated by a physician during delivery of the implantable device. The first and second securing structures may be part of a supporting structure for the tubular matrix. The securing structures are preferably constructed from radiopaque materials, such as include platinum, gold, silver, iridium, and the like. In addition, the first and second support structures preferably have a ring structure, which may have any suitable shape, preferably, a circular or elliptical shape.

The first and second securing structures may be secured by any suitable method known to those skilled in the art. For instance, the securing structures can be sutured to the matrix with a biocompatible suture material. Alternatively, the securing structures can be glued to the matrix. In another embodiment, the securing structures can be heat-bonded to the matrix, where the matrix or the securing structures have been pre-coated with a suitable heat-activated polymer or adhesive.

The deliverable device can be loaded onto an internal sheath, and the internal sheath carrying the deliverable device can itself be loaded into an external sheath of a delivery catheter. Suitable external sheaths for delivery of the occlusion device of the present invention can have an outer diameter from about 3 French to about 8 French, or from about 6 French to about 7 French. The dimensions of the sheathing will depend upon the dimensions of the occlusion device, and therefore in certain instances, the sheathing may be sized outside this range.

Support and Bias Behavior

Certain embodiments of the present invention may include a support structure for the matrix. The support structure may be an integral part of the matrix, or it may involve non-matrix elements, such as a frame that cooperates with the matrix when in use.

In one embodiment, the implant may comprise a matrix having a first orientation facilitating delivery of the implant to the interior of the aneurysm, and a second, concave orientation when the implant is in use in the interior aneurysm. The first orientation may comprise a number of regular and irregular shapes, such as, for example, a convex shape, a spiral shape, or any other non-concave shapes. The matrix of the implant may be at least partially supported by a support structure. In particular, the support structure (e.g., frame, frame arms or frame elements) of the implant is capable of adopting the first orientation facilitating delivery of the implant to the interior of the aneurysm, and the second, concave orientation when the implant is in use in the interior of the aneurysm. The concave orientation may have a degree of conformity to an interior surface of the aneurysm. That is, the concave orientation may complement the inner surface of the aneurysm. In embodiments in which the support structure comprises a frame, the frame may comprise one or more frame elements prepared from a biocompatible material which provides stability to the implant. The frame elements may, in some embodiments, be constructed from a shape memory material, that is, a material which “remembers” its geometry, and which regains its original shape after it has been deformed from its “original” conformation. An example of a biocompatible shape memory alloy is nitinol, a nickel-titanium alloy.

The frame can be prepared from other substances besides metals such as biocompatible polymers, which have shape memory and can be compressed into a narrow shape for delivery, and which can expand to adopt the desired geometry after insertion into the aneurysm sac. In one example, a “shape-shifting” biodegradable plastic may be composed of two components with different thermal characteristics, oligo(ε-caprolactone)diol and crystallisable oligo(p-dioxanone)diol to form a multiblock copolymer. The polymer has two block-building segments, a hard segment and a ‘switching’ segment, which are linked together in linear chains. Other examples of suitable biocompatible polymers are known to those of skill in the art.

The use of a shape memory material advantageously permits the physician to select a particular implant geometry, knowing that the pre-deployment and post-deployment dimensions will be approximately similar. Although the implant's frame will typically be extremely flexible to reduce the chances of damage to the aneurysm or vasculature, it will generally still be stiff enough to permit the frame to be adequately deployed to the intended area of treatment.

Instead of using a shape memory material, the frame may be constructed from a material which does not have shape memory. For example, the frame or frame arms may comprise elements which provide structural support for the frame but which do not themselves cause the implant to open or uncurl into a cup shape. For example, platinum filaments can be used as frame elements. In such an embodiment, the implantation device may be actively deployed by the physician, who would manually move the frame or frame arms from the collapsed condition to the expanded condition in the aneurysm sac. The frame arms may be connected to a central hub or ring via a chain link, hinge, or any other convenient mechanism which permits facile opening of the arms. Depending upon the particular circumstances, passive deployment such as blood flow in the sac may be used to expand the implant if a non-shape memory material is used.

If the frame is not radiopaque or readily visible by conventional medical imaging techniques, the frame elements may be plated or coated with a substance to provide for improved visibility. Such substances can include platinum, gold, silver, or iridium, as well as combinations of these or other materials. In addition, markers and/or structures constructed from radiopaque materials, such as include platinum, gold, silver, iridium, and the like, may be attached to the frame. The radiopaque markers and/or structures (e.g., filaments, coils) may be attached to the frame by any suitable means, for example, but not limited to, welding, soldering, suturing, threading, weaving the radiopaque marker and/or structures to the frame. The frame elements can also be woven or braided with another material to provide for enhanced radiopaque properties.

In certain embodiments, the frame or frame arms will adopt a generally cup-shaped form when they are in the fully-extended position. As previously stated, this cup-shaped form may be approximately in the shape of a disc, dish, ellipsoid, paraboloid, or hyperboloid, or any cup shape, when the arms are in the expanded condition. The expanded frame may have major and minor axes which are not identical in dimension, thereby yielding an ellipsoid structure. Complex memory shape for the frame arms can be used to provide a high degree of stability of the patch, especially in aneurysms with different sizes and shapes. The specific cup shape of the frame can be selected to fit and cover different anatomies of aneurysm neck or sac presented by individual patients.

In one embodiment, one end of each of the frame arms is coupled to a central hub or ring via a chain link, hinge, or any other convenient means, and the other ends of the frame arms are not joined together. After the implant is released from the delivery device, the frame arms move or are moved to their expanded position; the joined portions of the frame arms form the bowl of the cup shape and the unjoined portions of the frame arms form the opening of the cup. The frame arms can be coupled to the hub using an adhesive, solder, clip, hinge, spring, lock, or other suitable mechanism. Multiple coupling modes can also be used.

In another embodiment of the invention, the proximal and distal ends of the frame arms are joined at two corresponding central hubs, thereby obtaining a ball cage structure. The frame may have a mechanism which stretches the proximal and distal ends of the frame arms away from each other and thereby causing the ball cage to adopt an elongated or cylindrical compressed position. This elongated and collapsed position allows for insertion of the ball cage frame into the delivery device. Upon expansion of the delivery device in the aneurysm sac, a cup shape is adopted whereby the joined distal ends and joined proximal ends of the frame arms approach each other in 3-dimensional space. The resultant structure may resemble a rosette or deformed torus. Other designs having a cup shape according to this embodiment are possible and within the scope of the present invention.

The occlusion device, in some embodiments, comprises a plurality of frame arms. The number of frame arms can be varied, and there is no upper limit on the number of frame arms. For example, there may be two frame arms positioned at opposite ends of the frame or in relatively close proximity. In other embodiments of the invention, there may be 3, 4, 5, 6, 8, 10, 16, 30, 32, or more frame arms which form the occlusion device. There may be other numbers, even or odd, of the frame arms, and they may be evenly spaced apart or placed irregularly around the point(s) of attachment.

The frame arms do not all need to be identical or substantially identical, and the frame arms can have different dimensions. For example, the frame arms may have different lengths, widths, thicknesses, materials of construction, or have different spatial arrangement. In certain embodiments, there may two, three, or more sets of different frame arms, or each of the frame arms may have a different construction in order to provide the best fit into the aneurysm sac.

The frame arms can have any particular shape consistent with the ability of the frame to flip into the cup shape in the aneurysm. A non-limiting illustration of several frame arm shapes is provided in FIG. 6. For example, the arms can be single wires, spirals, logarithmic spirals or other spirally curled wires, curlicues, loops, strips, arms having loops or perforations, braided structures, or branched structures. The frame elements can have any suitable cross-section or diameter, although narrower elements will pass more readily through a patient's vasculature than wider elements.

In one embodiment, the frame elements are wires which have a diameter in the range of from 0.5 mil to 40 mil, such as from 1-10 mil. In other embodiments, the frame elements have a diameter which is outside of these ranges. The frame elements may have any kind of cross-sectional shape, such as circular, triangular, hexagonal, or elliptical, without limitation. The frame elements may also have a varying shape or dimension. For example, they may become narrower or finer at their free distal ends compared to the proximal ends.

In another embodiment of the invention, one or more of the frame arms may comprise at least in part a looped configuration. That is, the frame arm or a portion thereof may have a looped structure, or the entire frame arm itself may be a loop.

The specific shape or degree of curvature of the expanded implant device will largely depend upon the embodiment of the invention selected by the physician for use in a specific aneurysm. For an aneurysm having a wide neck and a large sac, a physician may choose a disc or plate-shaped implant, that is, one that has a shallow degree of curvature. For an aneurysm having a narrow neck but a deep sac, the physician may choose a fluted-shaped implant, one that has a high degree of curvature.

The frame of the occlusion device, or any portion of the frame, may be radiopaque to facilitate visualization of the device and its placement in the body by a medical imaging instrument. Platinum or another radiopaque material, or a marker of a size sufficient for detection, can also be incorporated into or onto the implant device to facilitate monitoring the deployment of the device and to aid in accurate placement within the target aneurysm. The matrix may also have a radiopaque component to aid in visualizing the implant. For example, radiopaque filaments may be incorporated into the matrix. Those of ordinary skill in the art will understand how to prepare the implant to permit its ready visualization in the body.

To facilitate radio-imaging, the implant may further comprise a radiopaque coil jacket formed from any suitable material, such as platinum or another biocompatible metal. The frame elements can be also constructed from different gauges of wires or other materials to provide different radial expansive force. As previously stated, the frame elements do not need to be identical, and different elements can have different dimensions or properties, such as length, width, thickness, and materials of construction.

The frame of the implantable device may be provided with a central aperture to facilitate insertion of an embolic agent into the aneurysm after deployment of the device. This central aperture may be a hub to which the frame elements are joined. The matrix of the device may also comprise a central aperture corresponding at least in part to the central aperture of the frame, thereby facilitating filling of the aneurysm. A controlled detachment mechanism, or a portion of it, may be provided at or near this location.

The implantable device may be constructed such that the frame has a first collapsed-umbrella position prior to implantation, and a second hyperextended-umbrella position following implantation. The flipped-umbrella position adopted by the frame elements may at least in part follow an interior contour of the aneurysm, thereby providing a close elastic but non-traumatic fit between the frame and the interior of the aneurysm such that the aneurysm wall is not damaged.

The frame can be collapsed to obtain a narrow profile for insertion into a delivery catheter or other device using any suitable techniques known in the art. In one embodiment, thermal setting is used to compress the device to fit into a delivery device. For example, the matrix may be collapsed or partially-collapsed by heating it between two plates, and then touching and fusing the frame arms to the heated matrix. Other modes of fitting the implantation device into a microcatheter or other delivery device are known to those of skill in the art.

The frame may be formed from any suitable biocompatible material. In one embodiment, the frame arms comprise a biocompatible metal or a polymer. Examples of biocompatible metals include nitinol, platinum, palladium, titanium, tantalum, and silver. Alloys and combinations of these or any other metals or polymers are within the scope of the invention. The frame arms or another portion of the device be radiopaque or may comprise a radiopaque material.

For implants comprising a shape memory material and having matrices with thin peripheral sections, the frame arms can bend back a substantial amount, giving the arms a spiral shape, such as a logarithmic spiral or a curlicue. In such embodiments, the spirals can provide a calibrated and constant expansion force, similar to a clockspring, which is perpendicular to the side of the aneurysm and which urges the implant to a tight fit with the walls of the aneurysm. The spirals can have any suitable dimensions. In one example, the spirals have a diameter of about 0.5 mm to about 5 mm.

The frame arms may also be adapted for controlled extension and retraction at any point between the expanded and collapsed positions. That is, the device may be constructed such that the frame arms are movable to any desired orientation or point between the fully-expanded and fully-collapsed positions.

Alternatively, the frame may comprise a number of regular and irregular shapes. Notably, the frame may have a spiral-shaped form when it is deployed into an aneurysm. The spiral-shaped frame may comprise at least one frame element having a spiral-shape. In one particular embodiment, the frame may comprise a plurality, (e.g., two or three) of parallel frame elements having a spiral-shape. The parallel frame elements are capable of receiving and supporting a matrix structure and providing a stabilized structure within the neck of the aneurysm.

The spiral-shaped form may be substantially flat, or may comprise of a three-dimensional spiral shape, where the interior spirals form a narrow base and the outer spirals form a wider top portion. The three-dimensional spiral shape may define a concave shape, such as a cup-shape, therein. The spiral shape may include for example, a logarithmic spiral, a Archimedean spiral, a conical helix, a hemi-spherical helix, and the like. The spiral-shaped frame can be manipulated to obtain a narrow profile for insertion into a delivery catheter or other device using any suitable techniques known in the art. In one embodiment, the spiral-shaped frame may be unwound and extended for insertion into a deliver device.

The implantable device may be constructed such that the frame has a substantially flat spiral shape prior to implantation, and a second three-dimensional spiral following implantation. The three-dimensional spiral of the frame may at least in part follow an interior contour of the aneurysm, thereby providing a close elastic but non-traumatic fit between the frame and the interior of the aneurysm.

### Matrix

The matrix may be formed from any kind of biocompatible material which serves to restrict or prevent movement of substances, such as an embolic agent or biological tissue, from the interior of the plugged aneurysm to the parent vessel. In one embodiment, the matrix comprises a porous foam such as a biodurable reticulated matrix which is permeable to blood or other bodily fluids and which allows cells or other biological tissues to access the interior surfaces of the implant.

In one embodiment, the matrix is a hydrophobic scaffold comprising a reticulated or substantially reticulated elastomeric polymeric matrix. The polymer matrix may be formed of a biodurable polymer which is resiliently compressible so as to regain its shape after being subjected to compression, for example, during delivery of the implant to the vascular deformation of other area of treatment. By suitable selection of the starting materials and processing conditions, the structure, morphology, and properties of the elastomeric matrix can be engineered for different uses or conditions.

In an embodiment of the present invention, the matrix is reticulated, that is, the matrix comprises a microstructure having an interconnected, intercommunicating, and continuous network of pores, voids, and channels that provide fluid permeability throughout the implantable device. This fluid permeability permits cellular and tissue ingrowth and proliferation into the interior of the implant.

In one embodiment of the invention, the matrix or scaffold is reticulated and permits blood or other bodily fluids to access interior surfaces of the implant. In another embodiment of the invention, the matrix is partially reticulated. In such an embodiment, the matrix may contain segments which are reticulated, and have pores for passage of fluids and ingrowth of tissues, and segments which are non-reticulated, and do not allow such passage of fluids or growth of tissues. For example, a reticulated segment may be separated from another reticulated segment by a non-reticulated segment. Any such combinations of reticulated and non-reticulated segments are encompassed by the present invention.

Generally, the properties of the matrix will be selected such that the implant has sufficient strength and biomechanical integrity for delivery to the target location without causing damage to the patient's vasculature while advancing the device in the body. If the matrix material is too stiff or too rigid, the implant will not flex or be deliverable to the aneurysm site through the vasculature without cause bodily damage.

In one embodiment, an inherently flexible matrix material can be used to form the implant so that the implant can pass through the tortuous contours of catheters placed in the human body, or that the implant can conform substantially to the internal shape and volume of the aneurysm sac. Alternatively, voids and cavities can be formed in a stiffer material to prepare the matrix and thereby provide a less rigid matrix. In an embodiment of the invention, if the matrix is formed from a viscoelastic substance which is not partially or substantially elastomeric, structural filaments need not be embedded or incorporated into the matrix.

In an embodiment of the invention, the matrix “scaffold” comprises a reticulated elastomeric polymeric material having sufficient structural integrity and durability to endure the biological environment for the intended period of implantation. In another embodiment, the scaffold comprises a partially or substantially reticulated elastomeric polymeric material. Alternatively, the matrix comprises a reticulated or partially reticulated viscoelastic polymeric material. The matrix may also comprise a hydrophobic or partially hydrophobic polymeric material.

In one embodiment a suitable matrix will be able to stretch with the frame or support structure as the device is opened from the collapsed or partially-collapsed position to hyperextended position to provide adequate coverage to the wall or neck of the aneurysm. In another embodiment, a suitable matrix will be flexible for folding into a cup shape and compressible for delivery into an aneurysm. The matrix can therefore be elastomeric in that it can be compressed for delivery and resiliently recover to substantially the pre-compression state. As an alternative to reticulated polymeric materials, other suitable substances include those that have pores or networks of pores that may or may not be interconnected and that permit biological fluids to have ready access throughout the interior of the implant. For example, woven or non-woven fabrics, or networked composites of microstructural elements may be used as the matrix.

In an embodiment of the present invention, the void phase of a reticulated elastomeric matrix used in the present invention may comprise range from about 20% to about 99% by volume of the matrix, such as about 50% of the matrix. The void volume refers to the volume provided by the interstitial spaces of the elastomeric matrix before an optional surface coating or layering is applied. In other embodiments, the void volume may range from about 70% to about 98% of the matrix, or from 80% to 98% of the matrix, or from 90% to 98% of the matrix. The void volume may also be outside of these ranges in particular embodiments of the invention. The void volume may also be continuous or non-continuous throughout the matrix, and different sections of the matrix may have different void volumes.

In this specification, the size or diameter of a spherical or substantially spherical pore is generally to be measured as its largest transverse dimension. The size of non-spherical pores, for example, ellipsoidal or tetrahedral pores or cells, is generally to be measured as the greatest transverse distance within the pore from one surface to another, e.g., the major axis of an ellipsoidal pore or the length of the longest side for a tetrahedral pore. Those of skill in the art will be knowledgeable in determining pore size or cell size of a reticulated matrix.

The cells of a reticulated matrix, such as a reticulated elastomeric matrix, may be characterized by their average cell diameter or by the largest transverse dimension of the individual cells forming the reticulated elastomeric matrix. The reticulated matrix comprises a network of cells which forms a three-dimensional spatial structure which is interconnected via the open pores therein. In one embodiment, the cells form a 3-dimensional superstructure. The pores are generally two- or three-dimensional structures. The pores provide connectivity between the individual cells, or between clusters or groups of pores which form a cell.

In one embodiment of the present invention relating to treatment of aneurysm or vascular malformation applications and the like, the role of the reticulated matrix is to encourage cellular ingrowth and proliferation and to provide adequate fluid permeability, the average diameter or other largest transverse dimension of pore size in a range from about 50 μm to about 600 μm. In another embodiment, the pores have an average pore size of from about 100 μm to about 500 μm. In still another embodiment, the pores have an average pore size of from about 150 μm to about 350 μm.

The cells of the matrix may have an average cell diameter or cell size in the range of from about 100 μm to about 1000 μm. In another embodiment, the cells have an average pore size of from about 200 μm to about 700 μm. In still another embodiment, the pores have an average pore size of from about 250 μm to about 650 μm. Alternatively, the cells of the matrix may have a size which is outside of any of these ranges.

In an embodiment of the invention, the pores of the matrix may be coated or filled with a cellular ingrowth promoter. In another embodiment, the promoter can be foamed or present as a film. The promoter can be any kind of biodegradable material that promotes cellular invasion of pores into the matrix in vivo. Promoters include naturally occurring materials that can be enzymatically degraded in the human body or are hydrolytically unstable in the human body, such as fibrin, fibrinogen, collagen, elastin, hyaluronic acid and absorbable biocompatible polysaccharides, such as chitosan, starch, fatty acids (and esters thereof), glucoso-glycans and hyaluronic acid. In some embodiments, the pore surface of the matrix is coated or impregnated.

In one embodiment, the matrix may be attached to and at least partially supported by the frame. For example, a matrix may have a cup-shaped, conical-shaped or spiral form that is attached to an partially supported by a circular, elliptical or spiral frame. In one embodiment, the frame may be sandwiched between two layers of the matrix material. This embodiment is particularly useful when the frame is in a spiral shape. In another embodiment, the a frame having parallel elements may Alternatively, the matrix may be attached to the frame and adopt the shape of the frame. Specifically, the matrix may extend partly or entirely up the length of the frame elements. That is, the matrix may be provided on a certain portion of the frame elements, such as on the lower half or lower two-thirds of the frame. The various frame elements may also have differing amounts of matrix affixed to their lengths. For example, one frame element may have the matrix coupled to its entire length, while another frame element in the same device may have the matrix only along half its length. In another embodiment of the invention, the matrix may extend over the entire frame to form a balloon which completely encapsulates the cup-shaped frame. Any such combinations of coverage are encompassed by the present invention.

The matrix can be attached by any suitable method known to those of skill in the art. For instance, the matrix can be sutured to the frame with a biocompatible suture material. Alternatively, the matrix can be glued to the frame. In another embodiment, the matrix can be heat-bonded to the frame, where the frame has been pre-coated with a suitable heat-activated polymer or adhesive. In another embodiment, a glue or suture is not used, and the matrix is partially or substantially mechanically wedged or “trapped” by the frame arm and the aneurysm wall.

In another embodiment, the matrix may be in a tubular form having a number of penetrations which in illustrated embodiments are in the form of slits, but which may be of other geometries as well. The tubular matrix may include a central aperture when arranged in a treatment configuration, or fitted within an aneurysm in a concave orientation having a degree of conformity to the interior of the aneurysm. In one embodiment, a central aperture of this type may comprise the inner lumen of the tubular matrix. Alternatively, the central aperture may comprise a separate penetration or set of penetrations of the occlusion device. As with other embodiments, but without limitation, the central aperture may (but need not) serve as a port for the delivery of filling structure or other material into the aneurysm.

The slits may be in any suitable, length, shape and/or arrangement. The slits may comprise straight cuts, or curved cuts, and thereby defining a plurality of matrix strips. The slits may be arranged such that they form circular, irregular, eccentric, oblong, bi-lobed, tri-lobed, or other shapes when the matrix is folded to a concave orientation. In one embodiment, the slits may be arranged such that the matrix forms a plurality of lobes. Preferably, the slits may be arranged such that the matrix forms between 2 to 100 lobes. More preferably, the slits may be arranged such that the matrix forms between 5 to 50 lobes. Most preferably, the slits may be arranged such that the matrix forms 10 to 25 lobes. In other embodiments, the slits may be arranged such that the matrix forms 2, 3, 4, 6, 8, 12, 16, 24, 30, 35, 40, or 48 lobes.

Preferably, the slits may be located at different sections along a longitudinal axis around the circumference of the tubular matrix. More preferably, the slits having the same shape and length may be grouped around a section of the tubular matrix. Several of these groups of slits may be formed in adjacent sections along the length of the tubular matrix in an offset pattern. The groups of slits in adjacent sections may be of different lengths, widths and/or shapes to optimize coverage of an opening.

The tubular matrix may be formed by cutting a tube from a large block of the matrix material. Alternatively the tubular matrix may be formed by rolling a continuous sheet of the matrix material to form a tube. The tube may be secured by any suitable method known to those skilled in the art. For instance, the matrix can be sutured to maintain the tubular shape with a biocompatible suture material. Alternatively, the matrix can be glued to maintain the tubular shape. In another embodiment, the matrix can be heat-bonded to maintain the tubular shape, where one edge of the matrix material has been pre-coated with a suitable heat-activated polymer or adhesive.

The matrix may be a semi-porous or porous material such as a biocompatible foam which supports an ingrowth of cells, and thereby plugging up the aneurysm and reducing the chances of rupture. The matrix may also be in the form of a web or a woven or non-woven fabric. The structure of the matrix of the invention may comprise interconnected networks of voids and/or pores encouraging cellular ingrowth of vascular tissue.

The dimensions and thickness of the matrix will depend upon the specific embodiment, and the matrix can have a uniform or non-uniform thickness. For example, the center of the matrix can be thicker or thinner than its periphery. In general, the thickness of the matrix affects how far the frame arms will bend back. Typically, the thinner the matrix is at its periphery, the more the arms can bend back. In an embodiment, the matrix is thin enough to permit the frame arms to curl and form a spiral shape. In one embodiment, the thickness of the matrix can range between 10 mil and 100 mil (0.25 mm-2.5 mm). In another embodiment, the matrix has a central portion which is about 100 mil thick, and a peripheral portion which has a continuously decreasing thickness from about 50 mil thick closer to the center to about 10 mil near the extreme periphery.

The matrix will generally have a substantial degree of flexibility, elasticity, or resilience to enable the implant to be compressed into the delivery device and to be expanded in the aneurysm sac without damage. The matrix will also generally be durable to prevent tearing, whether during delivery, after implantation in the aneurysm, or during cellular regrowth and aneurysm healing. The matrix may also have a degree of shape memory which serves to urge the implanted device to adopt a particular cup shape.

For embodiments of the present invention having a tubular matrix, the thickness of the matrix can affects how far the matrix will fold. The tubular matrix may preferably comprise a wall having variable thickness. Variable wall thickness may allow the tubular matrix to fold into specific geometries, which may include those that are more easily deliverable through a delivery device (e.g., microcatheter). In a specific embodiment, the matrix wall may be thicker in the center of the tubular matrix and thinner at the ends of the tubular matrix. The thinner matrix walls extending outward from the center provide a geometry that is less likely to damage the aneurysm or vasculature. Typically, the thinner the matrix is at its periphery, the more flexible it is and the less likely it is to damage the aneurysm or vasculature. In addition, a tubular matrix having a thinner may also provide improved conformity of the implant to an interior surface of the aneurysm. The tubular matrix will generally be durable to prevent tearing throughout its length, whether during delivery, after implantation in the aneurysm, or during cellular regrowth and aneurysm healing.

In embodiments of the present invention having a tubular matrix, the thickness of the matrix can be constant or variable. Preferably, the thickness of the matrix can range from about 0.005 inches (0.127 mm) to about 0.050 inches (1.27 mm). More preferably, the thickness of the matrix can range from about 0.015 inches (0.381 mm) to about 0.030 inches (0.762 mm).

The matrix may be formed from any kind of substance which provides the desired properties. In one non-limiting example, the matrix may be formed from collagen, fibronectin, elastin, hyaluronic acid, or a mixture of these or any other suitable substances. The matrix may be partly or wholly biodegradable.

The matrix can be biodurable or resorbable. In a particular embodiment, the matrix permits cellular ingrowth and proliferation into its matrix. In another particular example, the matrix is hydrophobic.

In another particular embodiment, the matrix includes an elastomer polymer. A list of non-exclusive examples of elastomer polymers includes polycarbonate polyurethanes, polyester polyurethanes, polyether polyurethanes, polysiloxane polyurethanes, polyurethanes with mixed soft segments, polycarbonates, polyesters, polyethers, polysiloxanes, polyurethanes. Alternatively, the matrix can include a mixture of two or more polymers, or a polymer and a non-polymer.

In still another embodiment, the matrix is reticulated and endoporously coated with a coating material that enhances cellular ingrowth and proliferation. In one example of the above embodiment, the coating material includes a coating, which can be a foamed coating, of a biodegradable material such as for instance, collagen, fibronectin, elastin, hyaluronic acid, or a mixture thereof.

In further embodiments of the invention, the matrix may include an elastomeric material. The elastomeric material can be a biodurable material, such as for instance, microporous expanded polytetrafluoroethylene. Alternatively, the elastomeric material can be a biosorbable material, such as but not limited to polyglycolic acid-polylactic acid copolymers.

In other embodiments, the matrix or portions thereof may be formed from polymers such as polyglycolic acid (“PGA”), polylactic acid (“PLA”), poly D-lactide, poly D-L lactide, polycaprolactic acid (“PCL”), poly-p-dioxanone (“PDO”), PGA/PLA copolymers, PGA/poly D-L Lacatide copolymers, PGA/PCL copolymers, PGA/PDO copolymers, PLA/PCL copolymers, PLA/PDO copolymers, PCL/PDO copolymers, or their mixtures and copolymers thereof, or combinations of any two or more of the above polymers.

Other suitable bioabsorbable materials can be solids, gels, or water-absorbing hydrogels with different bioresorption rates.

The matrix may optionally have a simple dip or spray polymer coating which facilitates preparation of the implant or which promotes cellular growth. The coating may comprise a pharmaceutically-active agent, such as a therapeutic agent or a drug. In one embodiment, the coating may be applied as a solution to the matrix. The polymer content in the coating solution may be in the range of from about 1% to about 40% by weight, or from about 1% to about 20% by weight. Alternatively, the polymer content in the coating solution may be from about 1% to about 10% by weight.

The film-forming polymer used to coat the matrix can optionally provide a vehicle for the delivery and/or controlled release of a pharmaceutically-active agent, for example, a drug. Examples of such embodiments are disclosed in the applications incorporated herein by reference. The pharmaceutically-active agent may be admixed with, covalently bonded to, or adsorbed in or on the coating of the matrix to provide a pharmaceutical composition.

The matrix itself may comprise a pharmaceutically-active agent. To form such matrices, the starting materials used to prepare the matrix may be admixed with the pharmaceutically-active agent prior to forming the matrix, or the pharmaceutically-active agent may be loaded into the matrix after it is formed.

Expandable materials can also be used as the matrix. Advantageously, these materials expand in the presence of a fluid such as blood or plasma to provide a tighter seal between the aneurysm and the implant device. Suitable expandable materials are known in the art, for example, hydrogels.

The matrix may also have a radiopaque component to aid in visualizing the implant. The matrix may also optionally include a radiopaque component to aid in visualizing the implant. A radiopaque material, or a marker of a size sufficient for detection, can be incorporated into or onto the matrix to facilitate monitoring the deployment of the device and to aid in accurate placement within the target aneurysm. Such radiopaque materials can include platinum, gold, silver, or iridium, as well as combinations of these or other materials. For example, radiopaque filaments may be incorporated into the matrix. The radiopaque filaments can also be woven or braided with the matrix. The addition of a radiopaque filament to various embodiments of the tubular matrix may provide an additional benefit of assisting the folding of the tubular matrix and/or maintaining the tubular matrix in the folded form. In one particular embodiment, slits of the tubular matrix are arranged such that the matrix forms a predetermined amount of lobes and the radiopaque materials are incorporated into at least one lobe at the apex of the lobe, thereby identifying the outer edges of the tubular matrix.

In one embodiment, the matrix may be a tubular matrix having radiopaque markers. The radiopaque markers can fluoroscopically outline the implant, in a compressed, folded or expanded state, when the implant is inserted into a patient. The markers may be placed: (1) at the ends of the tubular matrix; (2) along the length of the tubular matrix; and/or (3) within a number of matrix strips of the tubular matrix. The addition of a radiopaque filament to a tubular matrix may provide an additional benefit of assisting the folding of the tubular matrix and/or maintaining the tubular matrix in the folded form.

Additional examples and disclosure regarding suitable matrices are provided by WO 2004/062531, published Jul. 29, 2004 and WO 2004/078023, published Sep. 16, 2004. The contents of both publications are incorporated herein in their entirety by reference.

As previously discussed, an embolic agent may be used to fill or partly fill the aneurysm after deployment of the implant. The embolic agent may be any suitable substance or structural device known in the medical arts. For example, the embolic agent may comprise a solid material, such as an elastomeric matrix in the form of a string or other elongate form and having one or more structural filaments. Such structural filaments may comprise one or more platinum wires and polymeric fiber or filament. The elongate elastomeric forms may assume a non-linear shape capable of conformally filling the targeted aneurysm.

Examples of suitable solid embolic agents include Neuro-string™, developed by Biomerix Corp.; beads such as CelSphere®, marketed by Asahi Kasei America, Inc.; and embolic coils such as Nexus®, marketed by ev3 Inc. The solid embolic agent provides stability to the aneurysm so that healing can occur.

The embolic agent may also be a liquid substance, such as n-butyl cyanoacrylate polymer, which solidifies in the aneurysm to form a solid mass. Examples of liquid embolic systems include Onyx®, marketed by ev3 Inc.; and TruFill®, marketed by Cordis Corp. Liquid embolic systems are sometimes referred to as “glues”. The liquid embolic agents or glues can advantageously adopt the geometry of the aneurysm sac upon solidification and thereby provide an additional degree of safety for the patient. Liquid glues are also useful to stop bleeding aneurysms.

In addition, the solidified glue will typically surround and encapsulate the frame of the implant to form a single composite, thereby reducing the possibility that the implant will be ejected through the neck of the aneurysm. When the extremities of the frame arms project internally into the sac of the aneurysm, the liquid embolic agent behaves in a manner reminiscent of a rebar, thereby providing an additional degree of stability to the occlusion.

Combinations of different kinds of solid and/or liquid embolic agents are possible and are within the scope of the present invention. Selection of the specific embolic agent(s) will generally occur at the time of surgery at the direction of the physician.

According to another aspect of the invention, the use of an embolic agent allows for preparation of a composite embolic device for sealing the neck of the aneurysm. The structure of the embolic device at least partially surrounds the embolic agent, and the support elements such as frame arms are at least partially embedded in the embolic agent.

Delivery of the Occlusion Device

A delivery device is used to place the implant in the aneurysm sac. One or more connectors releasably connect the implant to the delivery device. After the delivery device has positioned the implant in the desired location, the connector releases the implant for permanent implantation in the body of the patient. The connector may be a single element or a plurality of elements which work in tandem, and may have any kind of convenient structure. In one embodiment, the connector comprises an internal or external thread. The connector may alternatively or additionally employ a coupling and detachment mechanism, as further discussed below.

The delivery device can have any particular dimensions so long as it can pass through the vasculature of a patient to deliver the implant to its ultimate position. In an embodiment of the invention, the delivery device may have an outside diameter in the range of from about 0.018 inch to about 0.040 inch. For example, the outer diameter of the delivery device (such as a microcatheter) can be 2 French (i.e. 0.026 inch/0.67 mm) or 3 French (i.e. 0.039 inch/1.0 mm). In another embodiment, the inside diameter of the delivery device may range from about 0.014 inch to about 0.021 inch.

In one embodiment, the delivery device may comprise a catheter such as an endoscope-guided catheter, wherein the endoscope assists in navigation of the catheter to the site of deployment. A guidewire may be located in the lumen of the device to facilitate delivery of the occlusion device.

The delivery device will typically be constructed to allow for a high degree of flexibility to navigate a patient's tortuous neuro-vasculature system. In one embodiment, this is achieved with a catheter of decreasing diameter from the proximal end (the end manipulated by the physician) to the distal end that delivers the implant into the sac of the aneurysm.

The delivery device may comprise a double lumen. In such an embodiment, an implant detachment wire may be located in the first lumen of the delivery device, and an embolic agent may be located in the second lumen of the device. Alternatively, the delivery device may have a single hollow lumen, and the guidewire and the embolic agent may be located in the single lumen. The lumen(s) may also be used for introduction of other substances or for placement of other wires or elements.

In one embodiment of the invention, the implant is structurally configured to be inserted over the tip of the delivery device in a ring-like manner, and has a surgical loop to facilitate controlled detachment of the implant from the delivery device.

### Controlled Coupling and Detachment of Occlusion Device and Delivery Device

One or more connectors can be used to releasably connect the occlusion device to the delivery device. The occlusion device is mounted or otherwise connected to the delivery device which transports the occlusion device to the target location. At the appropriate time, the physician will release the connector(s), thereby severing the link between the occlusion device and the delivery device. The occlusion device will thereby remain in the sac of the aneurysm after withdrawal of the delivery device.

The structure of the connector is not critical, and suitable connection mechanisms are known by those of ordinary skill in the art. Non-limiting examples of connection mechanisms are disclosed in the following applications: PCT/US2006/42357, filed Oct. 30, 2006; U.S. Ser. No. 11/229,044, filed Sep. 15, 2005; U.S. Ser. No. 11/111,487, filed Apr. 21, 2005; and U.S. Ser. No. 10/998,357, filed Nov. 26, 2004. The contents of these applications are incorporated by reference in their entirety.

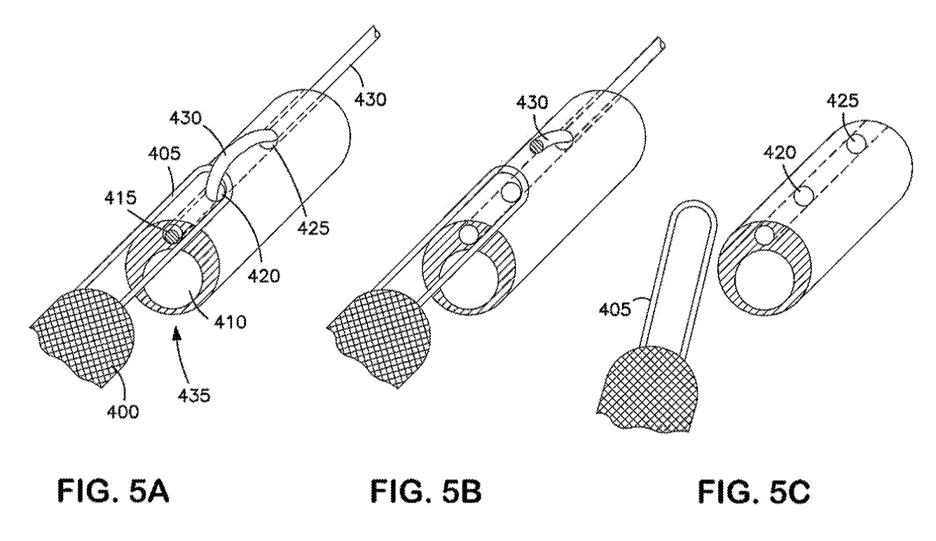

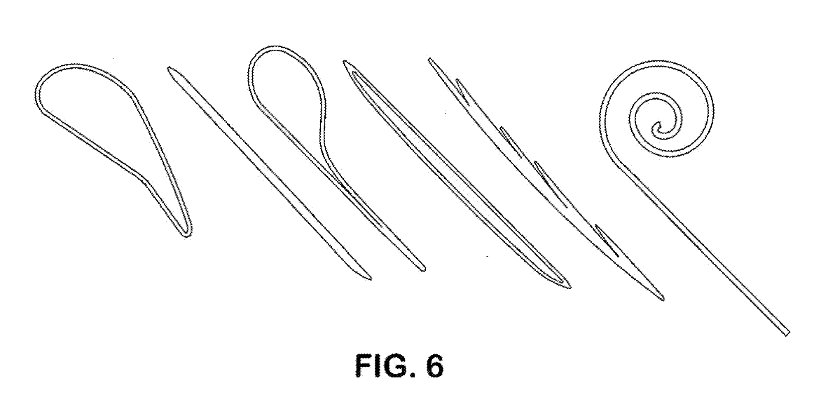

In one embodiment, a unitary coupling and detachment mechanism is used to releasably connect the occlusion device to the delivery device, as illustrated in FIGS. 5A-5C and further discussed below. This mechanism provides for controlled detachment of the implant in the target area. The mechanism also allows for a mechanically simple and rapidly-operated release of the implant without disturbing its precisely-selected position in the aneurysm.

In one embodiment, the implant bears a loop or other aperture-bearing engagement element. The loop is releasably held by a mating retaining element such as a detachment wire or guidewire. In some instances, an additional member can be used to constrain the engagement element relative to the retaining element.

A physician's actuation, such as causing one or more rotations or displacing the detachment wire, will lead to the withdrawal of the detachment wire from the loop, and thereby severing the connection between the implant and the delivery device. Release may be immediate, or near-immediate, and the loop is left behind as part of the implant with minimal or no disturbance to the aneurysm. Advantageously, the implant remains in place until such time that the implant is detached.

To facilitate attachment of the implant to the delivery device, the delivery device may have one or more side holes. In one embodiment, the delivery device may have a single side hole, and a portion of the detachment wire may pass through the side hole(s). In other embodiments, the delivery device has two side holes to facilitate attachment.

According to another embodiment of the invention, the implant may be engaged to the delivery device by a screw-threaded connector having an internal or external threading. To disengage the implant from the delivery device, the physician may rotate the delivery device or catheter, or a portion thereof, and thereby unscrewing the connection. After the delivery device is unscrewed, it is removed from the area of the aneurysm and withdrawn from the patient's body.

The invention will now be described with reference to the Figures, wherein like numerals indicate like elements.

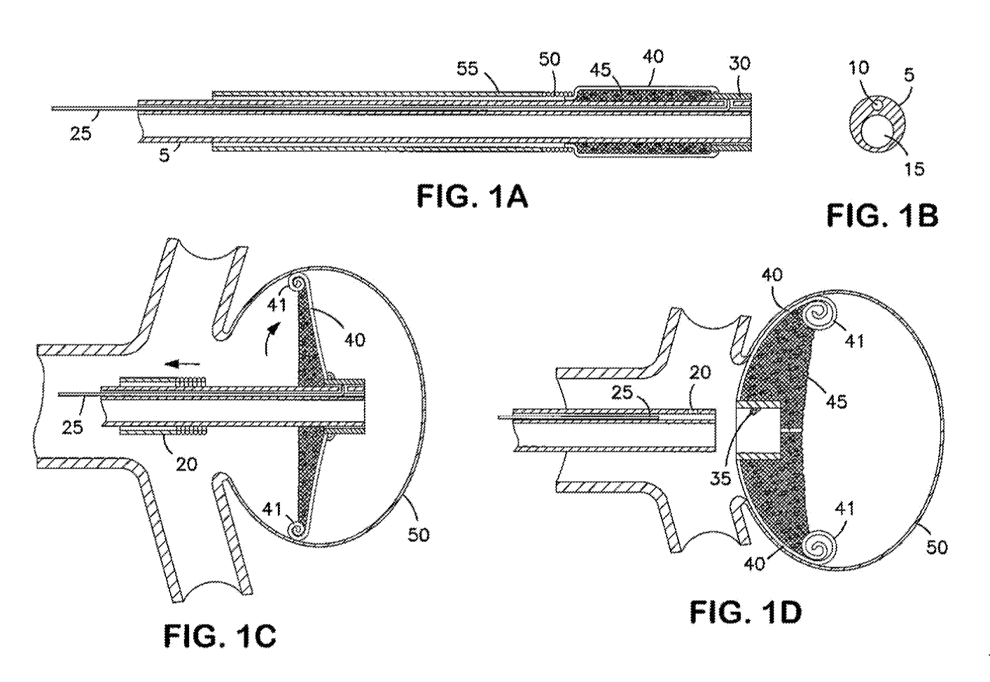

FIG. 1A illustrates a first embodiment of an implantable device according to the invention and shows an implantable device in the collapsed state and positioned over a delivery catheter. FIGS. 1C and 1D, respectively, show the device in partially expanded and fully expanded positions in an aneurysm. FIG. 1B shows a cross-section of a delivery catheter for use with the invention. The implantable device provides an expandable three-dimensional micro-structure implant positioned to seal the neck of an aneurysm.

As shown in FIG. 1A, prior to delivery to an aneurysm 50, the foldable frame arms 40 are collapsed into a low-profile by having all the arms 40 secured or inserted in the tip coil 50 of sheath 55. The frame arms 40 support a matrix 45 sutured to them. In this collapsed delivery position, the device can be navigated through a vasculature vessel into an aneurysm. In this embodiment of the invention, the implant device has a distal center ring or hub 30 which is positioned at the tip of the double-lumen delivery sheath 5. The delivery sheath 5 also has a main lumen 15 which may be used to pass an embolic agent into the aneurysm 50. A frame expansion sheath 55 is provided on the delivery sheath 5 to keep the arms of the implant collapsed during delivery to the target aneurysm, whether the arms have shape memory or not.

The device is secured in the closed position by a suture loop 35 and locked in this position using a core wire 25 via a side hole 20 in the detachment lumen 10 (depicted in FIG. 1B). Although only a single side hole 20 is illustrated to hold the core wire 25, there may be a plurality of side holes for this function. For example, the embodiment of the invention illustrated in FIGS. 5A-5C has two side holes.

In this embodiment of the invention, the frame arms are formed from nitinol which provides for a radial force expansion, and the implant has a platinum micro-coil jacket for radiopacity. Once released inside the aneurysm, the device opens from a collapsed condition to an expanded condition by the radial expansive force of the previously-compressed frame arms. In addition, the flow of blood in the aneurysm urges the frame arms to extend upward and outward and to adopt the expanded position. The frame arms 40 separate and move to the sides of the aneurysm to form a cupped shape or reversed umbrella shape closely fitting to the inside geometry of the aneurysm. The distal ends 41 of the frame arms 40 are seen to adopt a spiral orientation.

When the device is expanded, the frame arms 40 flip inside the aneurysm in the direction as shown by the arrows in FIG. 1C into a cup shape. The frame arms 40 are perpendicular to the wall of the aneurysm, and the curled portion 41 of the frame arms “point” into the internal portion of the implant frame. The spirals advantageously provide additional support and retention of the matrix as well as a force urging the implant against the walls of the aneurysm, thereby sealing the aneurysm from the vasculature.

The implantable device in FIG. 1C can be repositioned by moving the delivery sheath back and forward until the preferred position is reached to seal the neck of the aneurysm. This repositioning assists the frame arms in adopting their final fully-expanded condition. After the device is seated in the neck of the aneurysm, an embolic agent located in the main lumen of the delivery device may be used to fill the aneurysm. Suitable examples of embolic agents include elongate elastomeric members, coils, liquid glue, and other substances known in the art. The embolic agent fills the aneurysm cavity and thereby provides a radial force support to secure the device in an open position and seated across the neck of the aneurysm.

The matrix which is coupled to the frame arms provides an important safety feature by preventing the migration of materials from the filled or occluded aneurysm. Such a feature is particularly important when a glue injection is used as the embolic agent to prevent leakage into the patient's vasculature.

After the aneurysm is occluded with the embolic agent, the device can be controllably detached from the delivery catheter 5 by pulling the detachment core wire 25 to release the suture loop 35. Once the suture loop is free, the delivery sheath can be retracted and thereby releasing the implant in position inside the aneurysm.

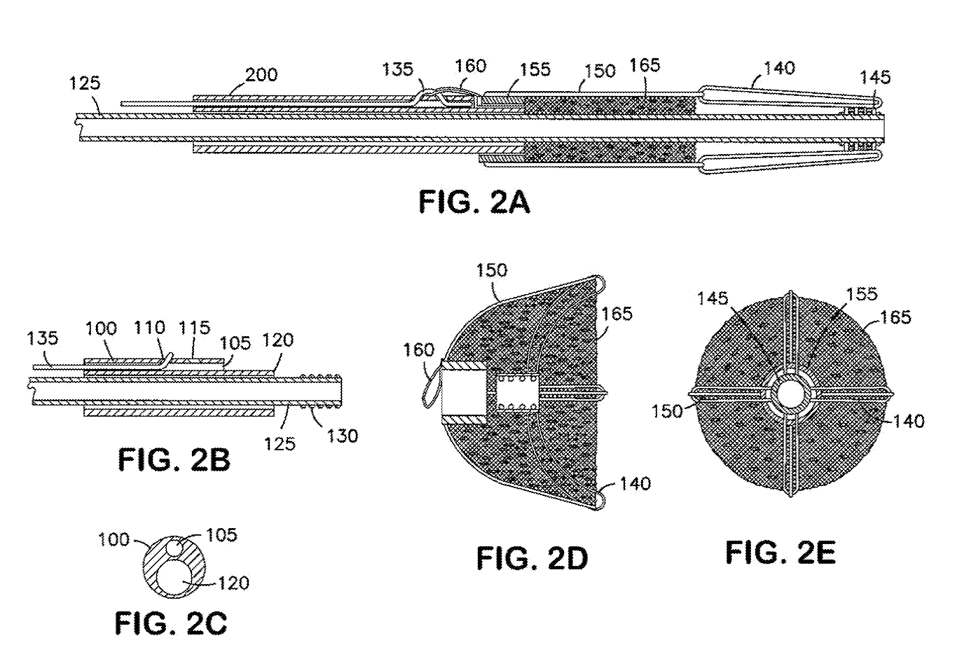

FIG. 2A illustrates a second embodiment of the invention showing an implantable device in the collapsed state. FIGS. 2D and 2E show views of the device in an expanded position. FIGS. 2B and 2C show views of a section of a delivery catheter used with the invention.

The embodiment of the invention illustrated in FIGS. 2A-2E contains four frame arms, each in the shape of a loop. The frame arms include a proximal section 150 and a distal section 140. Each frame arm is 90° apart. In different embodiments, there may be more or fewer frame arms which may or may not be equidistantly spaced apart. Each frame arm may also expand to a greater or lesser extent than other frame arms. A matrix 165 is affixed to the frame arms. The proximal section 150 of each frame arm is affixed to a central micro-ring 155, and the distal section 140 of each frame arm is affixed to another central micro-ring 145. The center micro-ring 145 of the implant has an internal thread-coil which engages with the tip-coil screw 130 of the micro-catheter 125, thereby releasably connecting the implant and the catheter.

The proximal arms 150 and distal arms 140 are connected like a chain link to provide easy hinging and flexibility of the frame during expansion or delivery to the intended area of treatment. The suture loop 160 and detachment core wire 135 form part of a coupling and detachment mechanism to provide an additional means of releasably connecting the implant and the delivery device.

During delivery, the frame is collapsed to form a low-profile and is engaged at the tip with the micro-catheter 125 to be navigated through the patient's vasculature. The proximal end of the frame is inserted at the tip of the delivery sheath 100 and secured by the suture loop 160 which is locked to the core wire 135. The double lumen delivery sheath 100 provides controlled delivery and controlled detachment of the implantable device. The small lumen 105 has a proximal side hole 110 and distal side hole 115 at the distal tip of the delivery sheath 100. The two side-holes facilitate positioning of the core-wire 135 out of the lumen 105, and lock the suture loop 160 by inserting the tip of the core wire 135 back into the lumen 105 through the distal hole 115.

In this embodiment, the expansion and collapsing of the frame is controlled by the tension of the micro-catheter. The tip of the micro-catheter is engaged with the frame similarly to a screw and nut connection. When the micro-catheter is pushed distally by using the guide wire (not shown), the frame remains in a collapsed position. If and when it is deemed necessary, the micro-catheter can be advanced from the frame by rotating distally and pushing forward to reach the required site and engaged back to the micro-screw 145 of the frame.

In contrast to the embodiment illustrated in FIGS. 1A-1D, which relies on the shape memory of the frame arms to expand the implant, the embodiment illustrated in FIGS. 2A-2E employs active means to expand the implant. That is, once the implant is properly positioned inside the aneurysm, the frame arms of the implant are mechanically expanded by the physician and flipped back or otherwise inverted into a cup shape by pulling the engaged micro-catheter back. This movement spreads the proximal and distal arms, and thereby causes the frame to adopt a pre-set or predetermined, generally cup-shaped geometry with a specific predefined diameter.

The distal arms will hinge or flip inside the opened proximal arms to move the frame into an expanded condition. The expanded frame has a cup-shaped configuration. The flipping movement can be reversed back and forward until a suitable position is reached to seal the neck of the aneurysm.

Once the frame is suitably seated in the neck of the aneurysm, the aneurysm can be filled with one or more embolic agents such as elongate elastomeric members, strings, coils, or a liquid glue injection through the micro-catheter lumen. The deployed embolic agents also fill the expanded frame and thereby provide an additional radial force support to keep the frame in an opened position in the aneurysm. The frame provides an important safety feature by preventing migration of agents during filling or occlusion of the aneurysm. This feature is particularly important if a glue injection is used to fill the aneurysm. Once the desired degree of controlled occlusion of the aneurysm has been obtained, the device is ready for controlled detachment from the delivery catheter.

In the embodiment illustrated in FIGS. 2A-2D, the frame has two connections to the delivery device: the suture loop 160, and the threaded microcatheter 130/micronut 145 combination. The first detachment is accomplished by unscrewing the tip of the micro-catheter 130 from the distal micro-nut 145 of the frame and by pulling it into delivery sheath 100. The second detachment is accomplished by releasing the suture loop 160 from the core wire 135 by pulling the core wire back. Once the suture loop has been freed, the delivery sheath can be pulled back and thereby leaving the implant inside the aneurysm. The mechanism of actuation of the suture loop is discussed in greater depth with regard to FIGS. 5A-5C.

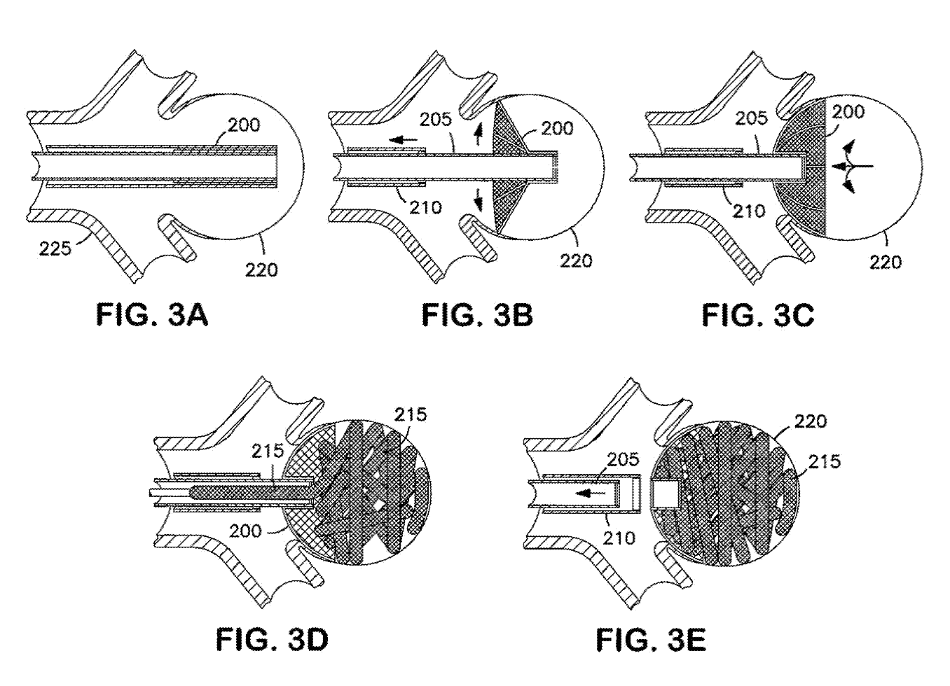

FIGS. 3A-3E illustrate various steps of a method of delivering an embodiment of the inventive device into an aneurysm of a patient. In FIG. 3A, the implant 200 is shown in a collapsed or folded position in the cavity of an aneurysm 220. The implant 200 is located over or on top of the tip of the delivery sheath 205. The tips of the frame arms are inserted into and locked by the detachment sheath 210 to prevent premature deployment. The collapsed position of the implant is narrow and thereby allows for its facile navigation through the vessels to access an aneurysm 220 of an artery 225. A matrix web is coupled to the frame arms.

FIG. 3B illustrates the deployment and expansion of the implant in the aneurysm using a passive deployment mechanism. The frame arms are formed from a shape memory material, and the arms expand when released from the delivery device. Once in place in the aneurysm, the implant can be expanded by pulling the detachment sheath 210 back. This action releases the frame arms of the implant 200 in the direction of the arrows so that they may open and expand like an umbrella inside the aneurysm sac behind its neck. The shape memory of the frame arms facilitates their expansion and adoption of a cup shape, for example, using a hinging mechanism such as a 90° hinge.

Although the compressed memory of the frame arms encourages expansion of the implant in the aneurysm sac, blood flow from the artery into the aneurysm further urges the expansion of the implant. The physician must choose the most appropriate sized implant. In this regard, the diameter of the expanded implant will generally be larger than the neck of the aneurysm in order to prevent migration of the implant out of the aneurysm and in the patient's vasculature.

FIG. 3C shows a “back-flip” of the implant which seals the neck of the aneurysm in accordance with the passive deployment or memory-activated deployment procedure. After the implant has been deployed and is in an opened-umbrella position in the sac of the aneurysm, the implant is flipped backwards (or placed in an inverted-umbrella position) by pulling the delivery sheath 205 back. This pull-back action causes the frame arms of the implant to fully expand. The flow of blood inside the aneurysm moves the frame arms outward and upward, and the frame arms flip inside against the wall of the aneurysm, thereby triggering formation of an internal cup which seals the neck of the aneurysm.

The implant or a portion thereof is radiopaque or visible under standard medical imaging procedures, permitting the physician to see the deployment and unfolding of the implant. In this embodiment, the frame arms are formed from nitinol, and the central ringed portion of the implant comprises a platinum coil jacket and a platinum center ring. In this embodiment, the matrix is a reticulated elastomeric polymeric material, although other suitable matrix materials can be used. Suitable materials will be readily apparent to those of ordinary skill in the art. Selection of the proper size and diameter of the implant will enable the matrix to provide soft, safe, and non-traumatic contact 360° around the open cup edge during the flip-action and thereby seal the aneurysm 220 from the artery 225.

FIG. 3D shows the filling of the sealed aneurysm 220 with an embolic agent 215. After the implant 200 is in a suitable position, a variety of embolic agents can be delivered into the inside of the sealed aneurysm. Examples of such embolic materials are coils, glue, or balloons. In one embodiment, the embolic agent is Neuro-string™, developed by Biomerix Corp.

FIG. 3E illustrates controlled detachment of the delivery device from the implant in the occluded aneurysm. Prior to delivery of the implant to the aneurysm, the delivery sheath 205 is screwed into the center ring of the implant 200 to secure the components for passage through the vasculature. Once total occlusion of the aneurysm is accomplished and the implant is in a stable position and has sealed the aneurysm neck, controlled detachment provides for release of the implant from the tip of the delivery sheath delivery sheath 205. The physician can readily un-screw the implant 200 while using the detachment sheath 210 compressed to the implant to ‘zero’ the pull of the implant, thereby minimizing chances that the implant will pop out of the aneurysm. The side-holes (illustrated in FIGS. 5A-5C) of the delivery sheath can be used for another method of detaching the implant from the delivery device.

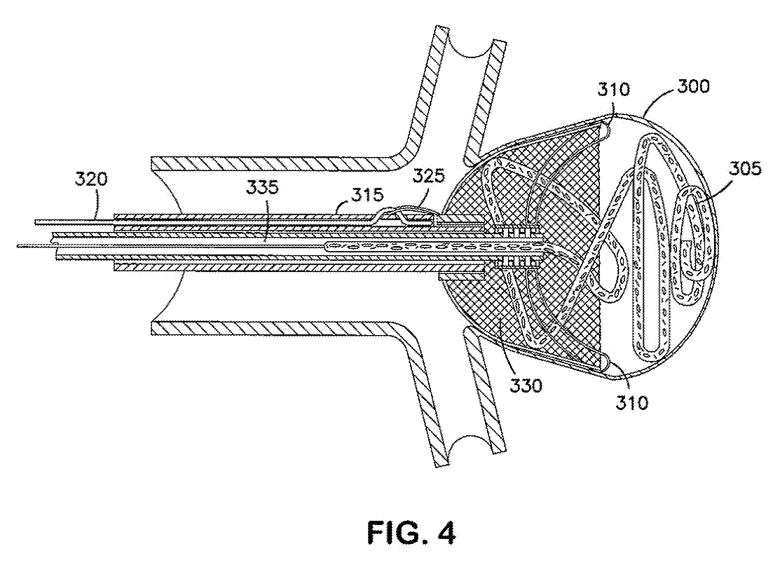

FIG. 4 illustrates further aspects of the embodiment of the invention shown in FIGS. 2A-2D which has been delivered into an aneurysm 300 and is in the process of being filled with a flexible longitudinally-extending elastomeric embolic agent 305. FIG. 4 also provides further illustration regarding the positioning of the implant during aneurysm occlusion with an embolic agent. The implant shown in FIG. 4 was opened from a collapsed position to an expanded position by active deployment. That is, the frame arms were mechanically caused to expand and flip into the illustrated cupped shape using the procedure described in relation to FIGS. 2A-2E.

As shown in FIG. 4, the implant is in an expanded position and has already been placed in the neck of the aneurysm 300 to provide a seal from the artery blood-flow. The implant has a plurality of frame arms 310, each in the shape of a loop, and a semi-porous matrix 330 affixed to the frame arms. The implant is attached to the delivery sheath 315 and to the tip of the internal micro-catheter 315. An embolic agent in the form of an elongate elastomeric member 305 is being deployed through the central lumen 335 of the micro-catheter 315 partially into the dome of the aneurysm and partially into the implant. A single portion of the embolic agent 305 may be delivered to the aneurysm. Alternatively, multiple portions of the embolic agent 305 may be delivered until the physician is satisfied with the degree of occlusion of the aneurysm 300. A pusher wire may be used to move the embolic agent 305 into the aneurysm sac. The embolic agent 305 passes from the central lumen 335 of the microcatheter delivery device 315 through the central aperture of the implant, and then into the cavity of the aneurysm.

Once the desired degree of partial or total occlusion is confirmed, the micro-catheter delivery sheath 315 can be un-screwed from the implant and pulled back into the delivery sheath. A second junction detachment takes place by unlocking the suture loop 325 using detachment core wire 320 to complete the separation of the cup-shaped implant from the delivery sheath 315.

FIGS. 5A-5C illustrate a double side-hole detachment mechanism which may be used to provide reliable attachment of an implant to a delivery device during delivery as well as controlled detachment of the implant on command.

FIG. 5A shows a surgical loop 405 of the implant 400 which is hooked or sandwiched between a core-wire 430 and the delivery sheath 435. The delivery sheath 435 has a larger lumen 410 and a smaller detachment lumen 415. The core wire 430 is inserted in the smaller detachment lumen 415 of the delivery sheath 435. The core wire 430 exits the lumen from a proximal detachment side-hole 425 and enters back through a distal detachment side-hole 420 to provide a secure attachment for the suture loop 405. If desired, additional surgical suture loops (not shown) can be used to provide for further retention of the implant by the delivery sheath.

FIG. 5B shows controlled detachment of the implant from the delivery device. The detachment core wire 430 is pulled back from the distal detachment side-hole 420, thereby providing instant or near-instant release of the hooked suture loop 405 from the delivery device.

FIG. 5C shows the total retraction of the detachment core wire 430 into the detachment lumen 415 and total separation of the implant 400 from the delivery sheath 435. At this point, assuming there are no other connections between the delivery catheter and the implant, the implant is fully installed and the delivery sheath can be retracted from the patient's body.

Additional embodiments of the detachment mechanism illustrated in FIGS. 5A-5C are provided by PCT/US2006/42357, filed Oct. 30, 2006, incorporated herein by reference in its entirety.

The type of detachment mechanism used in the present invention is not limited to those described above, and detachment structures other than those expressly described here are possible and within the scope of the present invention. For example, there may be only a single lumen within the delivery device, and this lumen may be used for both the detachment core wire and for transportation of the embolic agent into the aneurysm. Alternatively, there may be a single hole instead of two holes which is used for the detachment mechanism. Suitable detachment mechanisms are known to those of skill in the art.

FIG. 6 illustrates various embodiments of frame arms which may be used in connection with the present invention. For example, the arms can be single wires which are straight, curved, or curled into tight or loose spirals. The arms can also be branched, thereby facilitating retention of an embolic agent inserted or injected into the aneurysm sac. The frame arms may also be loops or comprise loops. All of such shapes and variations thereof are within the scope of the present invention.

The frame arms or a portion thereof may lie flat along the wall of the aneurysm to provide stability to the aneurysm. The frame arms may also be perpendicular to the wall of the aneurysm. Using the example in FIG. 1C of a spiral frame arm, the uncurled portion of the spiral may lie flat along the wall of the aneurysm, and the curled portion of the spiral may project into the lumen of the device.

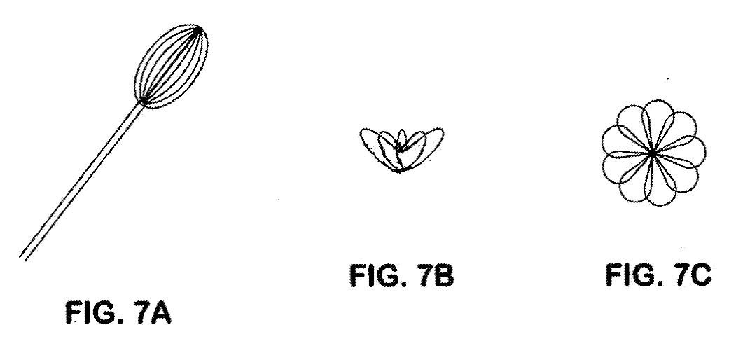

FIGS. 7A-7C illustrate an embodiment of the invention wherein the proximal and distal ends of the frame arms are joined at two corresponding central hubs. In FIG. 7A, the proximal ends of the frame arms are joined at a single point to a delivery device, and the distal ends of the frame arms are joined to another single point, thereby obtaining a ball cage structure. The frame has an internal mechanism, such as an internal wire, which allows the joined proximal and joined distal ends of the frame arms to be stretched away from each other, thereby placing the ball cage into an elongated and compressed condition. This elongated position allows for insertion of the ball cage frame into a delivery device for placement in the aneurysm sac.

FIG. 7B illustrates a perspective view of the implant after it has been released from the delivery device in the aneurysm sac and adopts its final shape. FIG. 7C shows a top view of the implant of FIG. 7B. As shown in FIG. 7C, the joined distal ends and joined proximal ends of the frame arms approach each other, or are caused to approach each other, and thereby allowing the implant to resume a cup shape. The resultant structure may resemble a rosette or deformed torus. Other designs having a cup shape according to this embodiment are possible and within the scope of the present invention.

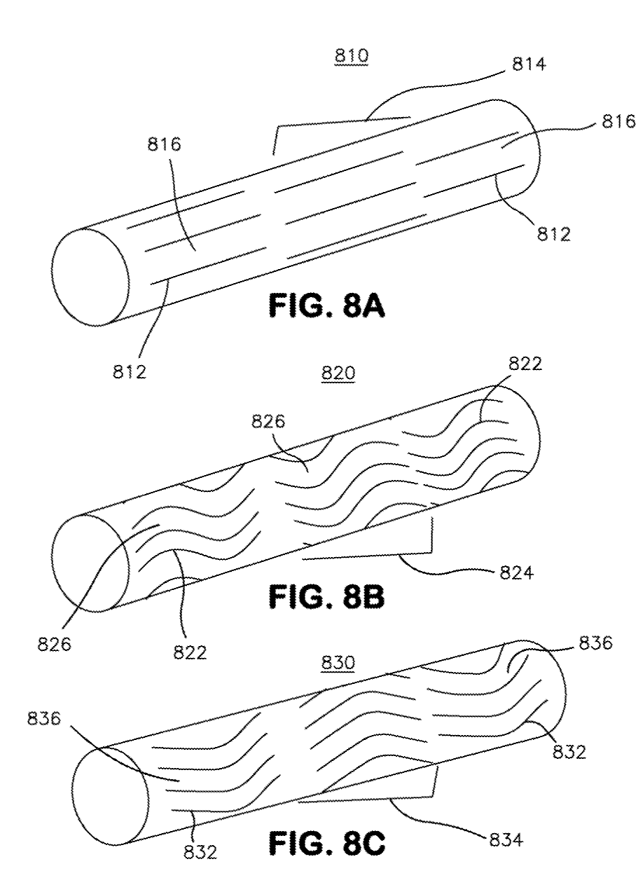

FIG. 8A illustrates an embodiment of a matrix 810 having a tubular form capable of folding into a cup-shape which may be used in carrying out the present invention. The tubular matrix 810 as shown in FIG. 8A is in an unfolded, expanded position. The exemplary tubular matrix 810 includes a plurality of linear, parallel slits 812 along a longitudinal axis around the circumference of the tubular matrix 810. The tubular matrix 810 includes a plurality of sections 814, wherein a plurality of linear, parallel slits 812 of the same length are cut therein. The slits 812 define a plurality of matrix strips 816.

FIG. 8B illustrates another embodiment of a matrix 820 having a tubular form capable of folding into a cup-shape which may be used in connection with the present invention. The tubular matrix 820 as shown in FIG. 8B is in an unfolded, expanded position. The exemplary tubular matrix 820 includes a plurality of curved, parallel slits 822 along a longitudinal axis around the circumference of the tubular matrix 820. The curved slits 822 of this particular embodiment has a sinusoidal shape. The tubular matrix 820 includes a plurality of sections 824, wherein a plurality of curved, parallel slits 822 of the same length are cut therein. The curved slits 822 define a plurality of curved matrix strips 826.

FIG. 8C illustrates another embodiment of a matrix 830 having a tubular form capable of folding into a cup-shape which may be used in connection with the present invention. The tubular matrix 830 as shown in FIG. 8C is in an unfolded, expanded position. The exemplary tubular matrix 830 includes a plurality of arched, parallel slits 832 along a longitudinal axis around the circumference of the tubular matrix 830. The tubular matrix 830 includes a plurality of sections 834, wherein a plurality of arched, parallel slits 832 of the same length are cut therein. The arched slits 832 define a plurality of arched matrix strips 836.

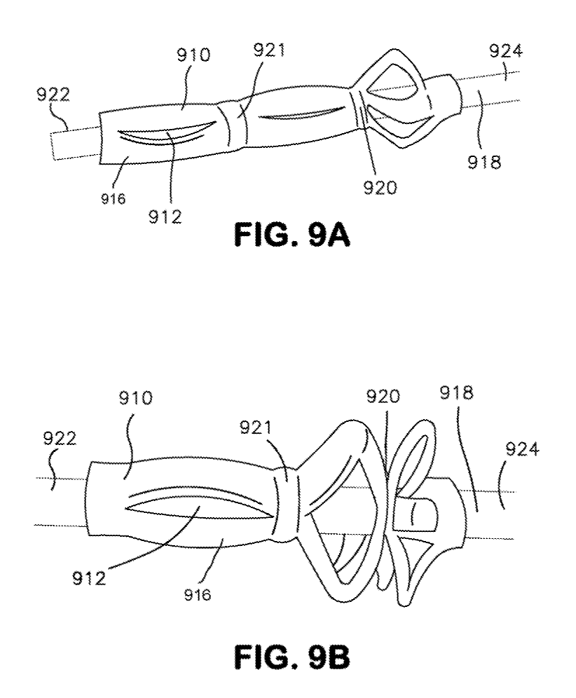

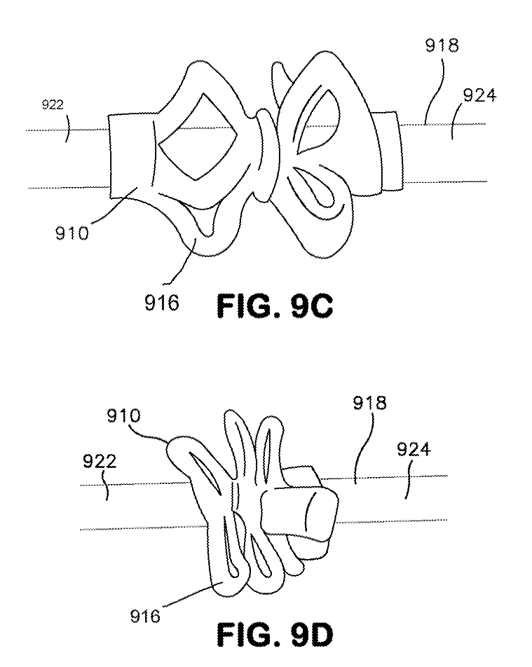

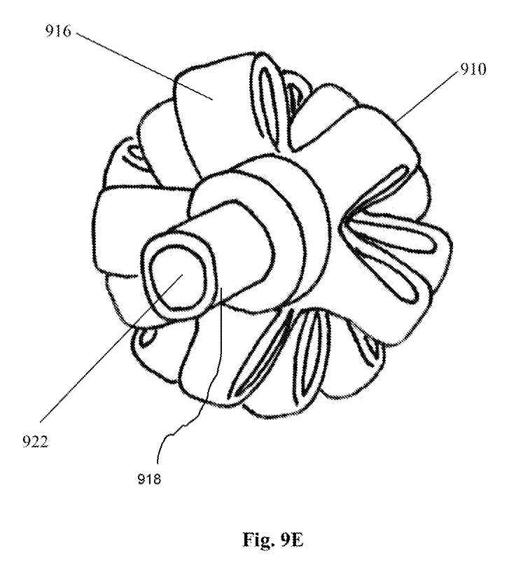

FIGS. 9A-9E illustrate various steps of a method of delivering an embodiment of the inventive device having a tubular matrix 910 into an aneurysm of a patient. In FIG. 9A, the implant is shown in an expanded, partially folded position fitted around a core wire 918. The proximal end 924 of the implantable device is partially pushed towards the distal end 922 of the implantable device, partially folding a portion of the tubular matrix 910. The implant comprises a tubular matrix 910 having a plurality of slits 912 along a longitudinal axis of its circumference, defining a plurality of matrix strips 916. The implant further comprises a plurality of support structures for securing the matrix 910 to the core wire 918. In one embodiment, the support structures surround the tubular matrix, forming a loop around and securing the matrix to the core wire.

Alternatively, the support structures may be secured to an interior surface of the tubular matrix, forming a loop around the core wire. The support structures may be secured to the matrix by any suitable method known to those skilled in the art. For instance, the support structures can be sutured to the matrix with a biocompatible suture material. Alternatively, the support structures can be glued to the matrix. In another embodiment, the support structures can be heat-bonded to the matrix, where the matrix or the support structures have been pre-coated with a suitable heat-activated polymer or adhesive.

FIG. 9B illustrates a first step for folding the tubular matrix 910 into a cup-shape. A first support structure 920 is pushed towards the distal end 922 of the implantable device and the matrix strips 916 between the first support structure 920 and the distal end 922 of the implantable device are extended outward from the core wire 918 in a curved and/or folded configuration, forming a first plurality of lobes.

FIG. 9C illustrates a second step for folding the tubular matrix 910 into a cup-shape. A second support structure 921 is then pushed towards the distal end 922. The matrix strips 916 between the first support structure 920 and the second support structure 921 are also extended outward from the core wire 918 in a curved and/or folded configuration, forming a second plurality of lobes. The second plurality of lobes may be aligned with or offset from the first plurality of lobes.

The inventive device may comprise more than two support structures. Additional support structures may be pushed towards the distal end 922 sequentially in a similar manner as illustrated in FIGS. 9B and 9C. It is contemplated that the tubular matrix 910 may also be folded by pushing the support structures in the opposite direction, towards the proximal end 924 of the inventive device.

FIGS. 9D and 9E illustrates a side view and a perspective view of the implant of FIG. 9A in a treatment configuration, respectively. The treatment configuration is a folded position fitted around a core wire 918. It is preferable that the lobes are offset from each other such that the tubular matrix bridges and/or occludes a neck of an aneurysm. The tubular matrix 910 is folded into a partially filled concave arrangement resembling that of a flower.

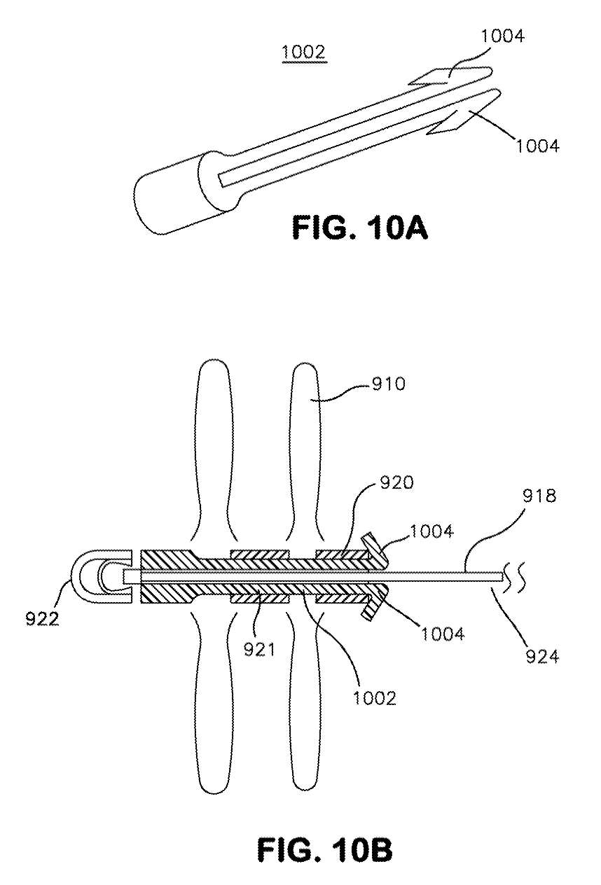

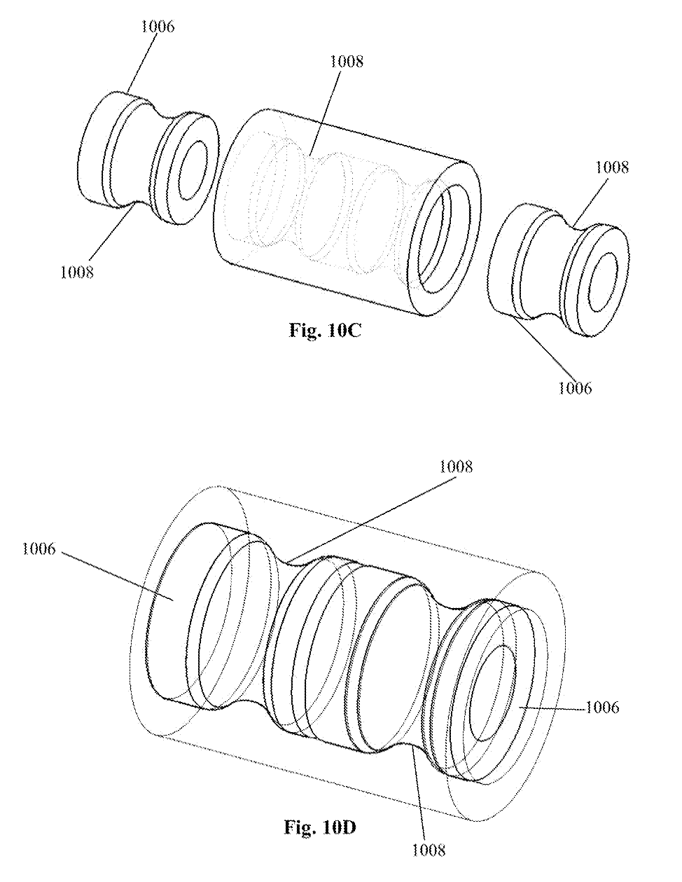

FIGS. 10A-10C illustrate two embodiments of a matrix folding mechanism which may be used to deliver an embodiment of the inventive device having a tubular matrix into an aneurysm of a patient.

FIG. 10A shows an embodiment of the matrix folding mechanism 1002 having outwardly extending prongs 1004. As shown in FIG. 10B, the prongs can engage the support structures of the implant of FIG. 9A and push the support structures towards the distal or the proximal end of the inventive device.

FIGS. 10C and 10D show an alternative embodiment of the matrix folding mechanism. As shown in FIG. 10C, the matrix folding mechanism is in a disconnected arrangement. In this exemplary embodiment, the support structures 1006 are tubular ring structures having an indentation at or about the center of the tubular ring structure. The support structures 1006 also function as a matrix folding mechanism. FIG. 10D shows the matrix folding mechanism in a connected arrangement. The indented portion 1008 of the support structure 1006 is capable of engaging an end of a neighboring support structure 1006 thereby attaching the two together.

FIGS. 11A and 11B, respectively show a side view and a perspective view of an embodiment of the inventive device having a conical matrix 1110. The apex 1112 of the cone is operably connected to a core wire 1118. The conical matrix 1110 as shown in FIGS. 11A and 11B is in a partially flipped, folded position. The conical matrix 1110 is partially inverted and folded across a horizontal plane perpendicular to the longitudinal axis of the cone.

While the invention has been particularly shown and described with reference to particular embodiments, those skilled in the art will understand that various changes in form and details may be made without departing from the spirit and scope of the invention.

We claim:

1. An implantable occlusion device for bridging the neck of an aneurysm, the device comprising:
a frame having a plurality of elements, the elements having a first pre-deployment position generally parallel to a major axis of a delivery lumen and having a first orientation, and a second post-deployment position spread radially from the major axis of the delivery lumen and having a second orientation; and
a matrix coupled to the frame.

2. The device according to claim 1, wherein the first orientation comprises a component direction substantially toward a proximal end of the lumen and the second orientation comprises a component direction substantially away from the proximal end of the lumen.

3. The device according to claim 1, wherein the frame is provided with a central aperture.

4. The device according to claim 3, wherein the matrix comprises a central aperture corresponding at least in part to the central aperture of the frame.

5. The device according to claim 4, wherein the central apertures are sized to permit passage of an embolic agent into the aneurysm.

6. The device according to claim 5, wherein the embolic agent passes from the delivery lumen into the aneurysm through an orifice of the frame.

7. The device according to claim 1, wherein the matrix comprises a porous foam or a reticulated foam.

8. The device according to claim 1, wherein the matrix comprises one or more substances selected from the group consisting of polycarbonate polyurethanes, polyester polyurethanes, polyether polyurethanes, polysiloxane polyurethanes, polyurethanes with mixed soft segments, polycarbonates, polyesters, polyethers, polysiloxanes, and polyurethanes.

9. The device according to claim 1, wherein the device is structurally designed and configured to be anchored in the neck of the aneurysm after delivery of an embolic agent into a sac of the aneurysm.

10. The device according to claim 9, wherein the device restricts migration of the embolic agent from the aneurysm into the parent vessel.

11. The device according to claim 1, wherein the device has between 3 and 32 frame elements.

12. The device according to claim 1, wherein the frame elements have a shape memory design which causes the device to expand from the pre-deployment position to the post-deployment position.

13. The device according to claim 12, wherein the frame arms in the post-deployment position comprise a spiral shape.

14. An implantable occlusion device for bridging the neck of an aneurysm, the device comprising a frame and a matrix, the frame having a first collapsed-umbrella position prior to implantation and a second hyperextended-umbrella position following implantation.

15. The device according to claim 14, wherein the frame comprises frame elements, and the hyperextended-umbrella configuration comprises the frame elements at least in part following respective aspects of an interior contour of the aneurysm.

16. The device according to claim 14, wherein the frame and the matrix each comprise a respective aperture, the respective apertures overlapping to permit insertion of an embolic agent into the aneurysm during a therapeutic treatment.

17. The device according to claim 14, wherein the matrix comprises a porous foam or a reticulated foam.

18. The device according to claim 14, wherein the matrix comprises one or more substances selected from the group consisting of polycarbonate polyurethanes, polyester polyurethanes, polyether polyurethanes, polysiloxane polyurethanes, polyurethanes with mixed soft segments, polycarbonates, polyesters, polyethers, polysiloxanes, and polyurethanes.

19. The device according to claim 14, wherein the frame comprises a material having a shape memory behavior which causes the device to restore from the pre-deployment position to the post-deployment position.

20. The device according to claim 14, wherein the frame comprises a material which lacks shape memory behavior causing the device to expand from the pre-deployment position to the post-deployment position.

21. The device according to claim 14, wherein the device is structurally designed and configured for passive deployment in a sac of the aneurysm.

22. The device according to claim 14, wherein the device is structurally designed and configured for active deployment in a sac of the aneurysm.

23. The device according to claim 14, wherein the device in the post-deployment condition has a diameter in the range about 2 mm and 20 mm.

24. The device according to claim 14, wherein the device in the post-deployment condition has a diameter in the range about 5 mm and 10 mm.

25. The device according to claim 14, wherein the device is structurally designed and configured to occlude a patient's vasculature.

26. The device according to claim 14, wherein the frame arms in the post-deployment position are in the shape of a spiral.

27. An apparatus for occluding the neck of an aneurysm, the apparatus comprising:
(a) an implantable occlusion device, the occlusion device comprising:
(i) a plurality of frame arms movable between a collapsed position prior to implantation and a generally cup-shaped position following implantation; and
(ii) a matrix coupled to the frame arms;
(b) a delivery device having a lumen, wherein the occlusion device is releasably mounted to a tip of the delivery device; and
(c) a coaxial detachment core wire located in the lumen of the delivery device.

28. The apparatus according to claim 27, wherein the lumen is structurally configured for delivery of an embolic agent therethrough.

29. The apparatus according to claim 28, wherein the embolic agent is a solid embolic agent or a liquid embolic agent.

30. The apparatus according to claim 29, wherein the embolic agent is a glue, coil, elongate elastomeric member, or a combination thereof.

31. The apparatus according to claim 27, wherein the occlusion device comprises a plurality of frame arms.

32. The apparatus according to claim 27, wherein the frame arms in the post-deployment position are in the shape of a spiral.

33. The apparatus according to claim 31, wherein one or more of the frame arms comprises at least in part a looped configuration.

34. The apparatus according to claim 27, wherein the frame arms comprise a biocompatible metal or a polymer.

35. The apparatus according to claim 27, wherein the frame arms comprise nitinol in combination with a metal selected from the group consisting of platinum, palladium, titanium, tantalum, silver, and alloys and combinations thereof.

36. The apparatus according to claim 27, wherein the least a portion of the occlusion device comprises a radiopaque material.

37. The apparatus according to claim 27, wherein the frame arms are flexible.

38. The apparatus according to claim 27, wherein the occlusion device is structurally designed and configured for passive deployment in a sac of the aneurysm.

39. The apparatus according to claim 27, wherein the occlusion device is structurally designed and configured for active deployment in a sac of the aneurysm.

40. The apparatus according to claim 27, wherein the frame arms are adapted for controlled extension and retraction at any point between the expanded and collapsed positions.

41. The apparatus according to claim 27, wherein the matrix is permeable or semipermeable to bodily fluids.

42. The apparatus according to claim 27, wherein the generally cup-shaped member is approximately in the shape of a section of a surface comprising at least one of a sphere, ellipsoid, paraboloid or hyperboloid, when the arms are in the expanded position.

43. The apparatus according to claim 27, wherein the matrix comprises a biocompatible foam which supports an ingrowth of cells.

44. The apparatus according to claim 27, wherein the matrix comprises a biocompatible foam comprising a substance selected from the group consisting of collagen, fibronectin, elastin, hyaluronic acid, and mixtures thereof.

45. The apparatus according to claim 43, wherein the matrix is biodegradable.

46. The apparatus according to claim 27, wherein the matrix comprises one or more substances selected from the group consisting of polycarbonate polyurethanes, polyester polyurethanes, polyether polyurethanes, polysiloxane polyurethanes, polyurethanes with mixed soft segments, polycarbonates, polyesters, polyethers, polysiloxanes, and polyurethanes.

47. The apparatus according to claim 27, wherein the occlusion device is releasably mounted to the delivery device by an internal or external thread.

48. The apparatus according to claim 27, further comprising a coupling and detachment mechanism, the mechanism comprising a loop structure coupled to the occlusion device, the mechanism configured to be coupled to the coaxial detachment core wire and to be released from the delivery device upon displacement of the core wire.

49. The apparatus according to claim 48, wherein a surface of the delivery device has at least one penetration adapted for receipt and controlled release of the core wire.

50. The apparatus according to claim 27, wherein the apparatus is used to seal a vasculature or a fistula of a patient.

51. A method for occluding an aneurysm, the method comprising the steps of
(a) providing the occlusion device according to claim 1, wherein the occlusion device is releasably attached to a delivery device having a lumen;
(b) positioning and deploying the occlusion device in the area of the aneurysm so that the device expands and thereby bridges and seals the neck of the aneurysm;
(c) delivering an embolic agent through the lumen of the delivery device into the aneurysm to secure the occlusion device; and
(d) detaching the delivery device from the occlusion device.

52. The method according to claim 51, wherein the step of delivering the embolic agent into the aneurysm causes the occlusion device to be anchored at the neck of the aneurysm.

53. The method according to claim 51, wherein the delivery device is a catheter or microcatheter.

54. The method according to claim 51, wherein the occlusion device is structurally designed and configured for passive expansion in a sac of the aneurysm.

55. The method according to claim 51, wherein the occlusion device is structurally designed and configured for active expansion in a sac of the aneurysm.

56. The method according to claim 51, wherein the occlusion device is attached to the delivery device by a double side-hole mechanism which provides for controlled detachment.

57. An implant for occluding the neck of an aneurysm, the implant comprising a surface having a first, convex orientation facilitating delivery to the interior of the aneurysm, and a second, concave orientation when in use in the interior of the aneurysm, the concave orientation having a degree of conformity to an interior surface of the aneurysm.

58. The implant according to claim 57, wherein the surface is provided with a bias urging it from a non-resting state in the first convex orientation to a resting state in the second, concave configuration.

59. The implant according to claim 58, wherein the surface comprises a frame, the flexure of which frame provides the bias to the surface.

60. The implant according to claim 59, wherein the frame comprises a plurality of coupled frame elements.

61. The implant according to claim 60, wherein at least some of the frame elements comprise elongate radially projecting elements.

62. The implant according to claim 61, wherein the radially projecting elongate frame elements have a resting state of spiral form.

63. The implant according to claim 62, wherein the spiral form comprises a logarithmic spiral form.

64. The implant according to claim 60, wherein, when the implant is in use within the aneurysm, a subset of at least some of the frame elements project at least in part toward the interior of the aneurysm.

65. The implant according to claim 57, wherein the surface of the implant, when in use, surrounds an embolic agent.

66. The implant according to claim 64, wherein the surface of the implant, when in use, surrounds an embolic agent, and the subset of at least some of the frame elements that project at least in part toward the interior of the aneurysm, project into the embolic agent.

67. The implant according to claim 57, wherein the surface comprises a porous biocompatible structure.

68. The implant according to claim 67, wherein the porous biocompatible structure comprises a reticulated foam.

69. A composite embolic device for sealing the neck of an aneurysm, the device comprising:
a structure comprising support elements having shape memory behavior,
the support elements' shape memory behavior comprising piecewise conformality to at least a portion of the aneurysm,
the structure at least partially surrounding an embolic agent, and
the support elements at least partially embedded in the embolic agent.

70. A device for treating an aneurysm wherein the aneurysm has an internal wall defining an internal volume, the device comprising:
an implant adaptable to
(1) a collapsed configuration for delivery to the aneurysm,
(2) an expanded configuration for delivery into the internal volume of the aneurysm, and
(3) a treatment configuration having at least one region of increased diameter greater than the diameter of the collapsed configuration, the region of increased diameter having at least one proximal face and at least one distal face;
the implant further adaptable to assume a treatment position within the aneurysm, in which position the at least one proximal face is convex.

71. The device according to claim 70, wherein, in the treatment configuration, the implant is an inverted-umbrella configuration.

72. The device according to claim 70, wherein, in the treatment configuration, the implant is in a generally cup-shaped configuration.

73. The device according to claim 70, wherein the at least one proximal face comprises a pre-selected number of proximal faces.

74. The device according to claim 70, wherein the at least one proximal face conforms at least in part to the internal wall of the aneurysm.

75. The device according to claim 70, wherein the implant comprises a compressible matrix having a pre-set shape memory, the matrix is compressed by application of a rotational force in the collapsed configuration and restored to the pre-set shape in the expanded configuration.

76. The device according to claim 70, wherein the implant further comprise a radiopaque marker.

77. The device according to claim 76, wherein the radiopaque marker is a filament comprising a radiopaque material.

78. The device according to claim 70, wherein the implant comprises a matrix.

79. The device according to claim 70, wherein the implant comprises a tubular shape.

80. The device according to claim 79, wherein the implant having a tubular shape comprises a matrix.

81. The device according to claim 80, wherein the tubular implant comprises a preselected number of apertures formed therein.

82. The device according to claim 78, wherein, in the expanded configuration, the implant has a tubular shape, and wherein the matrix comprises a number of slits.

83. The device according to claim 82, wherein, in the treatment configuration, the implant forms a predetermined number of lobes.

84. The device according to claim 83, wherein the implant forms at least two lobes.

85. The device according to claim 82, wherein the implant further comprises a frame operably attached to the matrix.

86. The device according to claim 85, wherein the frame comprises a plurality of frame elements, the frame elements capable of interlocking.

87. The device according to claim 85, wherein the frame comprises a plurality of frame elements, and wherein at least one frame element has a shape memory design which causes the implant to expand from the collapsed configuration to the expanded configuration.

88. The device according to claim 82, wherein the implant comprises a folding mechanism for manipulating the matrix from the expanded configuration to the treatment configuration.

89. The device according to claim 85, wherein the frame comprises a plurality of frame elements, and wherein the implant comprises a folding mechanism for manipulating the matrix from the expanded configuration to the treatment configuration, the folding mechanism sequentially engages the frame elements.

90. The device according to claim 70, wherein, in the expanded configuration, the implant has a conical shape.

91. An apparatus for occluding the neck of an aneurysm, the apparatus comprising:
(a) an implantable occlusion device, the occlusion device adaptable to:
(1) a collapsed configuration for delivery to the aneurysm,
(2) an expanded configuration for delivery into the internal volume of the aneurysm, and
(3) a treatment configuration having at least one region of increased diameter greater than the diameter of the collapsed configuration, the region of increased diameter having at least one proximal face and at least one distal face,
wherein the implant is further adaptable to assume a treatment position within the aneurysm, in which position the at least one proximal face is convex;
(b) a delivery device having a lumen, wherein the occlusion device is releasably mounted to a tip of the delivery device; and
(c) a coaxial detachment core wire located in the lumen of the delivery device.

92. The apparatus according to claim 91, wherein the lumen is structurally configured for delivery of an embolic agent therethrough.

93. The apparatus according to claim 92, wherein the embolic agent comprises at least one of a group consisting of a solid embolic agent and a liquid embolic agent.

94. A method for treating an aneurysm wherein the aneurysm has an internal wall defining an internal volume, the method comprising the steps of:
delivering an implant in a first, collapsed configuration to the aneurysm, wherein the implant is releasably attached to a delivery device;
expanding the implant into the internal volume of the aneurysm;
adapting the implant to a second configuration having at least one region of increased diameter greater than the diameter of the collapsed configuration, the region of increased diameter having at least one proximal face and at least one distal face, wherein the at least one proximal face is convex;
positioning the implant in the internal volume of the aneurysm for bridging and at least substantially sealing a neck of the aneurysm; and
detaching the delivery device from the implant.
